(19) United States
(12) Patent Application Publication
(10) Pub. No.: US 2021/0338174 A1
(43) Pub. Date: Nov. 4, 2021

(54) METHOD AND SYSTEM FOR ASSESSING EMERGENCY RISK FOR PATIENTS

(71) Applicant: KONINKLIJKE PHILIPS N.V., EINDHOVEN (NL)

(72) Inventors: MIRELA ALINA WEFFERS-ALBU, BOUKOUL (NL); PARASKEVAS DUNIAS, EINDHOVEN (NL)

(21) Appl. No.: 17/266,798

(22) PCT Filed: Aug. 2, 2019
(86) PCT No.: PCT/EP2019/070881

§ 371 Date: Feb. 8, 2021

Related U.S. Application Data
(60) Provisional application No. 62/716,517, filed on Aug. 9, 2018.

(51) Int. Cl. A61B 5/00; G16H 50/30; G16H 40/20; G16H 10/60; G16H 40/67; G16H 50/50; G16H 50/70; A61B 5/05; A61B 5/0205; A61B 5/08; A61B 5/01; G06T 7/13

(52) U.S. Cl. CPC A61B 5/7275; G16H 15/00; G16H 40/20; G16H 10/60; G16H 40/67; G16H 50/50; G16H 50/70; A61B 5/0082; A61B 5/05; A61B 5/0205; A61B 5/08; A61B 5/015; A61B 5/6892; A61B 5/0035; G06T 7/13; G16H 50/30

(57) ABSTRACT

A system for assessing emergency risk of patients in an emergency department is provided. The system includes a patient carrier comprising a transmissive structure, one or more optical or radar sensors configured to measure respiration information of the patient via light or radio waves traveling through at least a portion of the transmissive structure of the patient carrier, one or more additional sensors configured to measure injury information and cardiac information of the patient, and a computer system comprising one or more physical processors that are programmed with computer program instructions that, when executed cause the computer system to: determine an emergency risk parameter for the patient based on the cardiac information, the injury information and the respiration information of the patient obtained from the one or more sensors. The emergency risk parameter for the patient indicates that the patient requires medical intervention within a specified time period.

### BACKGROUND

### 1. Field

The present disclosure pertains to a method and a system for assessing emergency risk for patients, for example, at an emergency medicine facility.

### 2. Description of the Related Art

As indicated by the Agency for Healthcare Research and Quality in the U.S. (https://www.ahrq.gov/) “The purpose of triage in the emergency department (ED) is to prioritize incoming patients and to identify those who cannot wait to be seen. The triage nurse performs a brief, focused assessment and assigns the patient a triage acuity level, which is a proxy measure of how long an individual patient can safely wait for a medical screening examination and treatment. In 2008, there were 123.8 million visits to U.S. emergency departments. Of those visits, only 18% of patients were seen within 15 minutes, leaving the majority of patients waiting in the waiting room. The Institute of Medicine (IOM) published the landmark report, “The Future of Emergency Care in the United States,” and described the worsening crisis of crowding that occurs daily in most emergency departments. With more patients waiting longer in the waiting room, the accuracy of the triage acuity level is even more critical. Under-categorization (undertriage) leaves the patient at risk for deterioration while waiting. This is particularly relevant in the case of patient with cardiovascular risk as it can result in the patient death. Over-categorization (over-triage) uses scarce resources, limiting availability of an open ED bed for another patient who may require immediate care. And rapid, accurate triage of the patient is important for successful ED operations. Triage acuity ratings are useful data that can be used to describe and benchmark the overall acuity of an individual EDs' case mix. This is possible only when the ED is using a reliable and valid triage system, and when every patient, regardless of mode of arrival or location of triage (i.e. at the bedside) is assigned a triage level. By having this information, difficult and important questions such as, “Which EDs see the sickest patients?” and “How does patient acuity affect ED overcrowding?” can then be answered. There is also growing interest in the establishment of standards for triage acuity and other ED data elements in the United States to support clinical care, ED surveillance, benchmarking, and research activities.”

Also, in view of the sepsis risk and other complications associated with the delayed treatment of injuries, in general, it is imperative that patients who suffer fracture injuries are identified expeditiously and treated accordingly.

Strokes are cerebrovascular accidents, generally defined by the loss of brain function due to disturbance in the blood supply to the brain. In many situations, the disturbance is caused by blood flow obstruction in the cardiovascular system. As a result, an affected area of the brain cannot function, which results in inability to use one or more limb(s) (on one side of the body), inability to understand or formulate speech, and/or inability to see one side of the visual field (one-sided visual neglect/visual field loss). For that reason, strokes are medical emergencies that can cause permanent neurological damage and death. Given the impact to the patients' health as well as the associated costs of care, it becomes vital to find ways to predict, to detect early on, and to prevent the occurrence of a stroke.

According to 1) Sims N R, Muyderman H (September 2009). “Mitochondria, oxidative metabolism and cell death in stroke.” Biochimica et Biophysica Acta 1802 (1): 80-91. doi:10.1016/j.bbadis.2009.09.003) and 2) Donnan G A, Fisher M, Macleod M, Davis S M (May 2008). “Stroke”. Lancet 371 (9624): 1612-23. doi:10.1016/S0140-6736(08)60694-7, a stroke, or a cerebrovascular accident, is defined by the loss of brain function due to disturbance in the blood supply to the brain. The disturbance can be caused either by blood flow obstruction (obstructed blood vessel), or due to a haemorrhage (broken blood vessel). As a result of the blood supply disturbance, the affected area of the brain cannot function, which commonly results in 1) inability to make use of one's limb(s) (on one side of the body), 2) inability to understand or formulate speech, and/or 3) inability to see one side of the visual field (one-sided visual neglect/visual field loss). Stroke is a medical emergency and can cause permanent neurological damage and death.

According to http://www.uhnj.org/stroke/stats.htm, “stroke events claim on average worldwide, being the second leading cause of death, responsible for 4.4 million (9 percent) of the total 50.5 million deaths each year. In the US stroke is the 3^{rd} cause of death, behind heart disease and cancer. Stroke affects more than 700.000 new patients every year in the US, translating into that every 40 seconds a new stroke event will occur.” Stroke is also the leading cause of disability among adults in the United States to date. More than 4 million people in the United States have survived a stroke or brain attack and are living with the after-effects (http://www.uhnj.org/stroke/stats.htm). Currently, the U.S. healthcare system registers ˜3.500.000 rehabilitating patients. Current statistics of the impact of stroke in the U.S. are included below: 15 percent die shortly after the stroke, 40 percent of stroke victims experience moderate to severe impairments requiring special care, 10 percent of stroke victims require care in a nursing home or other long-term care facility, 25 percent of stroke victims recover with minor impairments, and 10 percent of stroke victims recover almost completely. Rehabilitation rates indicated in (Vilai Kuptniratsaikul et al., “First-Year Outcomes after Stroke Rehabilitation: A Multicenter Study in Thailand,” ISRN Rehabilitation Volume 2013 (2013), Article ID 595318, 6 pages) reveal a well-known fact that the rehabilitation success rates are low and they decrease with time: “First-Year Outcomes after Stroke Rehabilitation: the number of the patients who could function independently increased from 5.5% at discharge to 22.9% and 25.5% at month 6 and month 12, respectively. The change in functional ability level of 214 patients included improvement (51.5%), deterioration (12.8%), and equivocal (35.7%).” We include below some data and statistics giving insight into the stroke costs associated with stroke in U.S.: estimated cost of stroke—including rehabilitation—(in 2011) in U.S. was ˜78.9 B$/year. Costs within 30 days was estimated at $13.000 for mild strokes and $20 400 for severe strokes; reimbursement insurance companies costs: Hospital 16 B$/y; Nursing Home 15 B$/y; and lost Productivity was estimated at 14 B$/year. The rehabilitation process takes 5 weeks up to 12 months, while after 12-16 weeks increase is steepest. Reducing length of rehabilitation process by 10% entails a cost reduction of 0.7 B$/year for visual neglect only as an example. In the case of limbic impairment, the costs saved are higher given that the rehabilitation costs are higher and the impact on ability to work is more severe.

The current state of art provides tools for stratifying patients to identify those at risk for stroke. Those patients identified to be at risk are provided medication. Despite medication patients still experience strokes due to the fact that often their condition progresses and medication is not updated in time to keep up with deterioration. The disadvantage of this approach is that it does not enable patients to be aware when a stroke event is impending in order to seek specific medical care (which can include surgery) and thereby prevent the event.

The current state of art in triaging patients coming to the Emergency Department presents some disadvantages. For example, triaging is based on human assessment that can be subjective assessment. That is, the assessment can be incomplete, as the assessment does not involve scans to understand what happened internally (e.g., fractures, internal bleeding etc.). Also, the assessment requires time and expertise and the assessment can be incorrect.

### SUMMARY

Accordingly, it is an object of one or more embodiments of the present patent application to provide a system for assessing emergency risk of patients in an emergency department. The system includes a patient carrier comprising a transmissive structure. The patient carrier is configured to support a patient to lay over the transmissive structure of the patient carrier. The system also includes one or more optical or radar sensors configured to measure respiration information of the patient via light or radio waves traveling through at least a portion of the transmissive structure of the patient carrier. The system further includes one or more additional sensors configured to measure cardiac information of the patient and to determine injury information of the patient. The cardiac information of the patient includes blood pressure information of the patient, Electrocardiography (ECG) information of the patient and/or heart rate information of the patient. The injury information of the patient comprises information about size of the injury, information about position of the injury, and/or information about whether the injury is an internal injury or an external injury. The system also includes a computer system comprising one or more physical processors. The one or more physical processors are programmed with computer program instructions that, when executed cause the computer system to: determine an emergency risk parameter for the patient based on the cardiac information, the injury information and the respiration information of the patient obtained from the one or more sensors, the emergency risk parameter for the patient indicating that the patient requires medical intervention within a specified time period.

It is yet another aspect of one or more embodiments of the present patent application to provide a method for assessing emergency risk of patients in an emergency department. The method is implemented by a computer system comprising one or more physical processors executing computer program instructions that, when executed, perform the method. The method comprises obtaining, from one or more optical or radar sensors disposed in a transmissive structure of a patient carrier, respiration information of the patient, the patient carrier being configured to support a patient to lay over the transmissive structure of the patient carrier; obtaining, from one or more additional sensors, cardiac information of the patient, the cardiac information of the patient comprising blood pressure information of the patient, Electrocardiography (ECG) information of the patient and/or heart rate information of the patient; determining, using the one or more additional sensors (107a . . . 107n), injury information of the patient, the injury information of the patient comprising information about size of the injury, information about position of the injury, and/or information about whether the injury is an internal injury or an external injury; and determining, using the computer system, an emergency risk parameter for the patient based on the cardiac information, the injury information and the respiration information of the patient obtained from the one or more sensors, the emergency risk parameter for the patient indicating that the patient requires medical intervention within a specified time period.

It is yet another aspect of one or more embodiments to provide a system for assessing emergency risk of patients in an emergency department. The system comprises a means for executing machine-readable instructions with at least one processor. The machine-readable instructions comprise obtaining, from one or more optical or radar sensors disposed in a transmissive structure of a patient carrier, respiration information of the patient, the patient carrier being configured to support a patient to lay over the transmissive structure of the patient carrier; obtaining, from one or more additional sensors, cardiac information of the patient including blood pressure information of the patient, Electrocardiography (ECG) information of the patient and/or heart rate information of the patient; determining, using the one or more additional sensors (107a . . . 107n), injury information of the patient, the injury information of the patient comprising information about size of the injury, information about position of the injury, and/or information about whether the injury is an internal injury or an external injury; and determining an emergency risk parameter for the patient based on the cardiac information, the injury information and the respiration information of the patient obtained from the one or more sensors, the emergency risk parameter for the patient indicating that the patient requires medical intervention within a specified time period.

These and other objects, features, and characteristics of the present patent application, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present patent application.

### BRIEF DESCRIPTION OF THE DRAWINGS

FIG. 1 shows a system for assessing emergency risk for a patient in accordance with an embodiment of the present patent application;

FIG. 2 shows a system for emergency risk assessment for the patient in accordance with another embodiment of the present patent application;

FIG. 3 shows exemplary respiration patterns at rest interspersed with coughing events;

FIG. 4 shows exemplary respiration signal data (e.g., normal vs dyspnea);

FIG. 5 shows an exemplary right leg muscle injury;

FIG. 6 shows an exemplary system that uses thermal/IR camera and RGB cameras to pinpoint the location of an injury and to determine whether the injury is an internal injury or an external injury in accordance with an embodiment of the present patent application;

FIG. 7 shows an exemplary rib fracture (i.e., internal injuries do not involve external hemorrhage or wounds);

FIG. 8 shows an exemplary fractured left femur;

FIG. 9 shows an exemplary system that uses sound waves to scan an injury site in accordance with an embodiment of the present patent application;

FIG. 10 shows a method for assessing emergency risk of patients in an emergency department in accordance with an embodiment of the present patent application;

FIG. 11 shows exemplary ECG pattern characteristic of myocardial infraction;

FIG. 12 shows exemplary PQRST wave;

FIG. 13 shows exemplary ECG signal data (e.g., at moderate hyperkalemia) showing distinct characteristic features;

FIG. 14 shows exemplary ECG QRS signal data pattern (e.g., at mild, moderate and severe hyperkalemia);

FIG. 15 shows exemplary ECG sinusoidal signal data pattern (e.g., at final stage hyperkalemia);

FIG. 16 shows exemplary ECG signal features characteristic for each hyperkalemia phase;

FIG. 17 shows exemplary ECG signal wave changes at hypokalemia compared to the normal ECG signal wave at normokalemia; and

FIG. 18 shows exemplary ECG signal features characteristic for each hypokalemia phase.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, the term “or” means “and/or” unless the context clearly dictates otherwise.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present patent application provides an automatic solution for the assessment of emergency risk of patients coming to the Emergency Department. The solution provides an objective assessment that is correct and complete (in the aspects it monitors). The solution also allows internal scanning and supports the triage process/procedure enabling patients to be treated within optimal time within optimal medical staff resources.

The present patent application proposes an early diagnosis for fast Emergency Department (ED) triage and during emergency response. In addition, the system of the present patent application is configured to provide an automatic assessment of the healing process that is useful in taking fast measures in case of internal bleeding after surgery or onset of inflammation or infection.

In some embodiments, a system 100 for assessing emergency risk of patients in an emergency department. System 100 includes a patient carrier 202 comprising a transmissive structure. Patient carrier 202 is configured to support a patient to lay over the transmissve structure of patient carrier 202. System 100 also includes one or more optical or radar sensors 106a . . . 106n. In some embodiments, one or more optical or radar sensors 106a . . . 106n may be embedded in the transmissive structure of patient carrier 202. One or more optical or radar sensors 106a . . . 106n are configured to measure respiration information of the patient via light or radio waves traveling through at least a portion of the transmissive structure of patient carrier 202. System 100 further includes one or more additional sensors 107a . . . 107n configured to measure cardiac information of the patient and determine injury information of the patient. The cardiac information of the patient includes blood pressure information of the patient, Electrocardiography (ECG) information of the patient and/or heart rate information of the patient. The injury information of the patient comprises information about size of the injury, information about position of the injury, and/or information about whether the injury is an internal injury or an external injury. System 100 also includes a computer system 102 that comprises one or more physical processors. The one or more physical processors are programmed with computer program instructions which, when executed cause the computer system to: determine an emergency risk parameter for the patient based on the cardiac information, the injury risk information and the respiration information of the patient obtained from the one or more sensors. The emergency risk parameter for the patient indicates that the patient requires medical intervention within a specified time period.

Emergency department, as used herein, refers to any medical treatment and health care facility that specializes in emergency medicine. Emergency department may also be referred to as accident and emergency department, emergency room, and casualty department. In some embodiments, emergency department may be part of a hospital system. In some embodiments, system 100 is also be used in an urgent medical treatment and health care facility. In some embodiments, system 100 is also used in other primary care medical treatment and health care facilities.

Triage, as used herein, refers to a process/procedure of determining the priority of patients' treatments based on the severity of their condition. For example, triage may result in determining the order and priority of emergency treatment. Triage may also result in the order and priority of emergency transport, or the transport destination for the patient. Triage also rations treatment to the patients efficiently when available resources are insufficient to treat all the patients immediately.

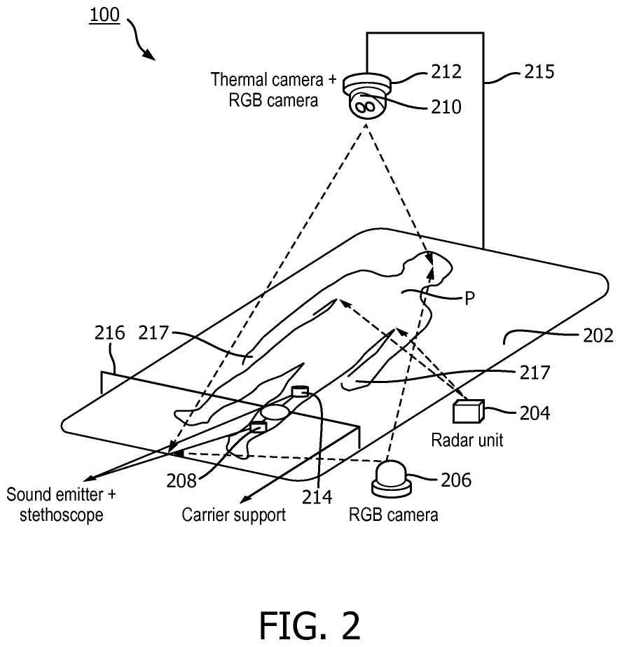

As will be clear from the discussions below, in some embodiments, system 100 includes computer system 102 that has one or more physical processors programmed with computer program instructions that, when executed cause computer system 102 to obtain information or data from one or more sensors 106a . . . 106n; 107a . . . 107n associated with patient P or with patient carrier or support 202 (see FIG. 2). In some embodiments, patient carrier or support 202 is configured to support patient P in the emergency department.

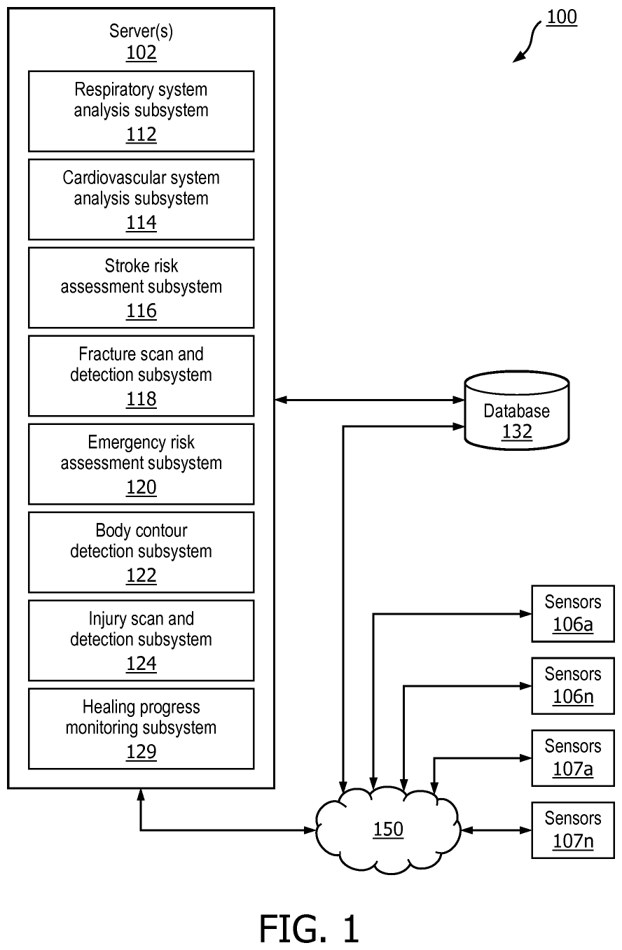

As shown in FIG. 1, system 100 for assessing emergency risk of patients in the emergency department may comprise server 102 (or multiple servers 102). In some embodiments, server 102 may comprise respiratory system analysis subsystem 112, cardiovascular system analysis subsystem 114, injury scan and detection subsystem 124, emergency risk assessment subsystem 120 or other components or subsystems. In some embodiments, server 102 may comprise respiratory system analysis subsystem 112, cardiovascular system analysis subsystem 114, fracture scan and detection subsystem 118, emergency risk assessment subsystem 120 or other components or subsystems. In some embodiments, server 102 may comprise respiratory system analysis subsystem 112, cardiovascular system analysis subsystem 114, stroke risk assessment subsystem 116, fracture scan and detection subsystem 118, emergency risk assessment subsystem 120, healing progress monitoring subsystem 129, body contour detection subsystem 122, injury scan and detection subsystem 124, or other components or subsystems. In some embodiments, server 102 may comprise respiratory system analysis subsystem 112, cardiovascular system analysis subsystem 114, stroke risk assessment subsystem 116, injury scan and detection subsystem 124 and/or fracture scan and detection subsystem 118, emergency risk assessment subsystem 120 or other components or subsystems.

In some embodiments, system 100 is configured to assess injury risk parameter of patients in the emergency department. In some embodiments, injury risk assessment system comprises body contour detection subsystem 122, injury scan and detection subsystem 124, emergency risk assessment subsystem 120, or other components or subsystems. In some embodiments, system 100 may also include healing progress monitoring subsystem 129 that is configured to assess the healing progress of an injury detected and treated earlier.

In some embodiments, system 100 is configured to assess fracture risk parameter of patients in the emergency department. In some embodiments, fracture risk assessment system comprises body contour detection subsystem 122, injury scan and detection subsystem 124, emergency risk assessment subsystem 120, fracture scan and detection subsystem 118, or other components or subsystems.

In some embodiments, body contour detection subsystem 122 is configured to detect the contour of the patient body including the position of head, limbs and, in particular, palms. In some embodiments, respiratory system analysis subsystem 112 is configured to monitor and determine the status of the respiratory system of the patient. In some embodiments, cardiovascular system analysis subsystem 114 is configured to monitor and determine the status of the cardiovascular system of the patient. In some embodiments, stroke risk assessment subsystem 116 is configured to monitor and determine the status of the patient's stroke risk. In some embodiments, fracture scan and detection subsystem 118 is configured to detect fractures in the patient. In some embodiments, injury scan and detection subsystem 124 is configured to detect internal and/or external injuries/wounds of the patient. In some embodiments, emergency risk assessment subsystem 120 is configured to assess the emergency risk of a patient based on input provided by all (or a subset of) subsystems described above and assist in patient triage at the emergency department.

In some embodiments, stroke risk assessment subsystem 116 is configured to monitor and determine the stroke risk assessment of the patient. In some embodiments, stroke risk assessment subsystem 116 is configured receive data from a wearable device attached to the patient neck, positioned over an artery. In some embodiments, stroke risk assessment subsystem 116 is configured to monitor, using an optical sensor, the blood flow volume through the artery. In some embodiments, stroke risk assessment subsystem 116 is configured to compare the patient blood flow parameters with baseline values of healthy individuals of similar profile as the patient. In some embodiments, significantly diminished blood flow at rest increases the chance that the patient is at risk (or may have recently undergone) a stroke event. In some embodiments, the stroke risk is higher the lower the blood flow parameter is in comparison with the baseline blood flow at rest for a similar population of patients.

In some embodiments, stroke risk assessment subsystem 116 includes a device is attached to the patient neck (e.g., on top of an artery). In some embodiments, stroke risk assessment subsystem 116 is configured to assess, in real-time, the patient risk for suffering stroke. In some embodiments, stroke risk assessment subsystem 116 is also configured to trigger accordingly an alarm when the risk for stroke increases over time. In some embodiments, stroke risk assessment subsystem 116 is configured to monitor the blood flow through the artery during defined tasks such as: 1) during walking, 2) during climbing stairs, 3) while getting out of bed, 4) while getting up from a chair. In some embodiments, stroke risk assessment subsystem 116 is configured to perform the following: 1) compare the blood flow parameters during these activities with baseline values of healthy individuals of similar profile as the patient, and 2) monitor over time blood flow parameters during activities above in order to determine whether decreasing trends can be established indicating a progressively obstructed artery which raises the risk of a stroke incident. In some embodiments, based on the trend values and slope, stroke risk assessment subsystem 116 is configured to calculate a stroke risk that quantifies the probability of a stroke and triggers an alarm to the patient's medical staff.

In some embodiments, the wearable device of stroke risk assessment subsystem 116 includes 1) an accelerometer—required in order to be able to detect patient activities of interest (e.g., walking, climbing stairs, getting out of bed, getting out of chair); and 2) an optical sensor that is used in order to monitor the blood flow volume through the artery.

In some embodiments, stroke risk assessment subsystem 116 is configured to analyze signals received from the sensors incorporated in the wearable device above and calculate/determine a stroke risk score/parameter. In some embodiments, stroke risk assessment subsystem 116 is configured to generate triggers alarms based on the determined stroke risk score/parameter.

In some embodiments, stroke risk assessment subsystem 116 includes an activity detection subsystem that is configured for automatic detection of 1) walking; 2) climbing stairs; 3) getting out of bed; and 4) getting up from a chair. In some embodiments, stroke risk assessment subsystem 116 includes a blood flow assessment subsystem that is configured to determine blood flow parameters via the optical sensor embedded in the device. In some embodiments, stroke risk assessment subsystem 116 includes an analytics subsystem that is configured to analyze the blood flow parameters trends during specific activities (indicated above) and calculating in real-time a stroke risk. In some embodiments, stroke risk assessment subsystem 116 includes a feedback subsystem configured to provide reports and triggering alarms based on the data trends established as well as the values and evolution of the stroke risk.

In some embodiments, the activity detection subsystem of stroke risk assessment subsystem 116 is configured to automatically detect patient's activities by analysing and classifying the accelerometer signal sent out from the wearable device. In some embodiments, automatic detection of the activities above is done via machine learning algorithms.

In some embodiments, the blood flow assessment subsystem is configured to measure blood-flow volume in subcutaneous tissue by placing the device in contact with the patient neck, placed over an artery. As explained in

(https://global.kyocera.com/news/2016/1205_nvid.html): “when light is reflected on blood within a blood vessel, the frequency of light varies—called a frequency or Doppler shift—according to the blood-flow velocity. In some embodiments, the sensors of the device of stroke risk assessment subsystem 116 utilizes the relative shift in frequency (which increases as blood flow accelerates) and the strength of the reflected light (which grows stronger when reflected off a greater volume of red blood cells) to measure blood-flow volume.”

In some embodiments, the analytics system of stroke risk assessment subsystem 116 is configured to analyze the blood flow parameters trends during specific activities (indicated above) and to determine, in real-time, a stroke risk parameter/score. This is done as follows: each time one of the activities above is detected, stroke risk assessment subsystem 116 records the blood flow volume put out by the blood flow assessment subsystem during that activity. In some embodiments, the analytics subsystem then compares the most recent values to previous values collected during the same activity in the past and analyses trends. In some embodiments, the stroke risk score is calculated based on a formula that combines the information regarding the blood flow volume levels and evolution during each of the activities of interest:

StrokeRiskScore=Slope_BloodFlowVolume_Walk⊗Slope_BloodFlowVolume_Stairs⊗Slope_BloodFlowVolume_BedExit⊗Slope_BloodFlowVolume_ChairExit Equation (D)

Slope_BloodFlowVolume_Walk in equation (D) represents the blood flow Volume trend slope during walking. Slope_BloodFlowVolume_Stairs in equation (D) represents the blood flow volume trend slope during climbing stairs. Slope_BloodFlowVolume_BedExit in equation (D) represents the blood flow volume trend slope during getting out of bed. Slope_BloodFlowVolume_ChairExit in equation (D) represents the blood flow volume trend slope during getting out of a chair.

In some embodiments, decreasing trends (negative slope values) imply a narrowing of the artery over time, which increases the stroke risk.

In some embodiments, the stroke risk score/parameter is determined using Equations (E), (F) and (G).

StrokeRiskScore=−1*mean(Slope_BloodFlowVolume_Walk, Slope_BloodFlowVolume_Stairs, Slope_BloodFlowVolume_BedExit, Slope_BloodFlowVolume_ChairExit) Equation (E)

StrokeRiskScore=−1*min(Slope_BloodFlowVolume_Walk, Slope_BloodFlowVolume_Stairs, Slope_BloodFlowVolume_BedExit, Slope_BloodFlowVolume_ChairExit) Equation (F)

StrokeRiskScore=−1*(weight_walk*Slope_BloodFlowVolume_Walk+weight_stairs*Slope_BloodFlowVolume_Stairs+weight_BedExit*Slope_BloodFlowVolume_BedExit+weight_ChairExit*Slope_BloodFlowVolume_ChairExit) Equation (G)

In some embodiments, the various weights in equations (E), (F), (G) are always positive values assigned based on an assessment of each activity relevance regarding increasing blood volume through the artery. In some embodiments, the weights in equations (E), (F), (G) can be learned for each patient by observing the blood flow volume during the four activities: the activity producing the highest blood flow volume is assigned the highest weight value, etc.

In some embodiments, the feedback subsystem of stroke risk assessment subsystem 116 is configured to provide reports and triggering alarms based on the data trends established as well as the values and evolution of the stroke risk. As an example, prior to a stroke occurring, there is generally a gradual process of physiological deterioration. Stroke risk assessment subsystem 116 may generate one or more alerts related to the physiological deterioration (or other alerts) over time. As such, by the time a stroke occurs, stroke risk assessment subsystem 116 may have already issued a number of alerts indicating the heightened risk of impending stroke event. In one use case, such alerts may be transmitted to the whole infrastructure of care around the patient (e.g., specialist, GP, home care provider, and emergency services for input). Thus, in a further use case, when the patient is checked into the emergency department, the emergency department system (e.g., via stroke risk assessment subsystem 116) already has feedback regarding the heightened patient stroke risk data and the alarms/alerts, and such feedback can be used to support the diagnosis of the patient.

In some embodiments, cardiovascular system analysis subsystem 114 is configured to obtain cardiac information and respiration information of the patient from the one or more sensors. In some embodiments, the cardiovascular risk parameter for the patient indicates that the patient requires medical intervention within a specified time period. In some embodiments, cardiovascular system analysis subsystem 114 is configured to monitor and determine the cardiovascular system status of the patient. In some embodiments, cardiovascular system analysis subsystem 114 is configured to determine the cardiovascular distress. The cardiovascular risk parameter is determined using wearable ECG and blood pressure sensors applied to patient wrist, allowing measurement of heart rate, along with the analysis of the PQRST complex. Cardiovascular distress is determined based on: 1) myocardial Infarction risk assessment subsystem; 2) hyperkalemia risk assessment subsystem; and 3) hypokalemia risk assessment subsystem. The cardiovascular risk score/parameter is based on the risks assessed above.

In some embodiments, system 100 includes 1) myocardial infarction risk assessment subsystem, 2) hypokalemia risk assessment subsystem, 3) hyperkalemia risk assessment subsystem, and 4) cardiovascular system analysis subsystem 114. In some embodiments, the myocardial infarction risk assessment subsystem, the hypokalemia risk assessment subsystem and the hyperkalemia risk assessment subsystem are configured to monitor and determine the respiratory system status of patient P. In some embodiments, cardiovascular system analysis subsystem 114 is configured to monitor and determine the cardiovascular system status of patient P. In some embodiments, cardiovascular distress is determined based on: myocardial infarction risk assessment by myocardial infarction risk assessment subsystem, hyperkalemia risk assessment by hyperkalemia risk assessment subsystem, and hypokalemia risk assessment by hypokalemia risk assessment subsystem.

In some embodiments, as will be clear from the discussion below, computer system 102 is further configured to: determine a myocardial infraction risk parameter for the patient using the blood pressure information of the patient obtained from the blood pressure sensor, the Electrocardiography (ECG) information of the patient obtained from the Electrocardiography (ECG) sensor, the respiration information of the patient obtained from the respiration sensor and the heart rate information of the patient obtained from the heart rate sensor, determine a hyperkalemia risk parameter for the patient and a hypokalemia risk parameter for the patient using the Electrocardiography (ECG) information of the patient obtained from the Electrocardiography (ECG) sensor; and determine the cardiovascular risk parameter for the patient using the determined myocardial infraction risk parameter, the determined hyperkalemia risk parameter, and the determined hypokalemia risk parameter for the patient.

In some embodiments, the myocardial infarction risk assessment subsystem is configured to detect the following symptoms to calculate a myocardial infarction risk parameter using a) non-typical values of heart rate and blood pressure, b) ECG characteristic patterns and c) dyspnea. In some embodiments, the heart rate information, the blood pressure, the ECG characteristic patterns of the patient are obtained from the heart rate sensor, the blood pressure and the ECG sensor, respectively. In some embodiments, the dyspnea information is obtained from the respiration sensor, respectively.

In some embodiments, the myocardial infarction risk assessment subsystem is configured to analyze the heart rate information and the blood pressure information of patient P (obtained from the heart rate sensor and blood pressure sensor, respectively) to determine a risk of an impending myocardial infraction. For example, the myocardial infarction risk assessment subsystem is configured to determine a risk of an impending myocardial infraction using 1) whether the obtained heart rate information is above a predetermined threshold, 2) whether the obtained heart rate information is below a predetermined threshold, 3) whether the obtained blood pressure information is above a predetermined threshold, or 4) whether the obtained blood pressure information is below a predetermined threshold.

In some embodiments, non-typical values of heart rate and blood pressure are configured to increase the risk of an impending myocardial infarction. In some embodiments, elevated heart rate values increases the risk of an impending myocardial infarction. In some embodiments, depressed heart rate values increases the risk of an impending myocardial infarction. In some embodiments, the heart rate is generally measured by the number of contractions of the heart per minute (beats per minute, bpm). In some embodiments, elevated blood pressure values increases the risk of an impending myocardial infarction. In some embodiments, depressed blood pressure values increases the risk of an impending myocardial infarction. In some embodiments, the blood pressure is generally measured in millimeters of mercury (mm Hg).

In some embodiments, the heart rate is generally in the range of between 60 and 70 beats per minute. In some embodiments, the heat rate is generally elevated when the heart rate is more than 100 beats per minute. In some embodiments, the heart rate is generally depressed when the heart rate is less than 60 beats per minute.

In some embodiments, the blood pressure is generally in the range of between 90/60 and 120/80. In some embodiments, the blood pressure is generally elevated when the blood pressure is more than 120/80. In some embodiments, the blood pressure is generally elevated when the blood pressure is more than 140/90. In some embodiments, the blood pressure is generally depressed when the blood pressure is less than 90/60.

In some embodiments, the myocardial infarction risk assessment subsystem is configured to analyze the ECG characteristic patterns of patient P (obtained from the ECG sensor) to determine a risk of an impending myocardial infraction. For example, the myocardial infarction risk assessment subsystem is configured to determine a risk of an impending myocardial infraction by detecting the presence of the pathological Q waves, and ST elevation or ST depression.

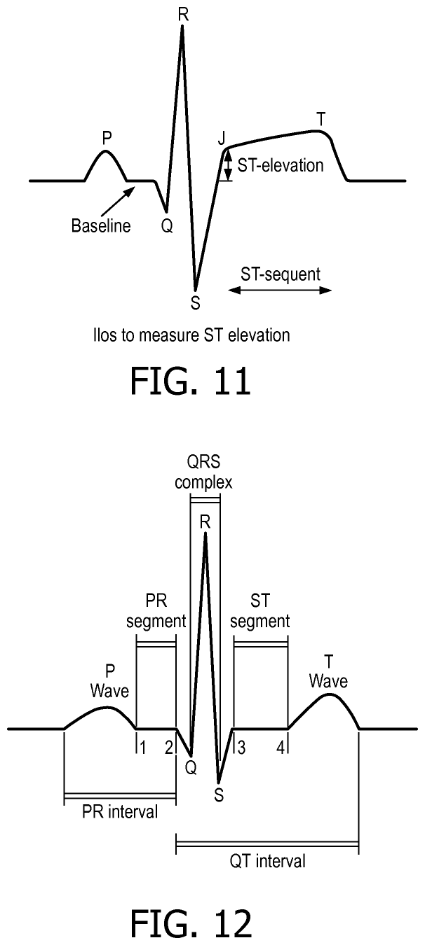

FIG. 11 shows ECG patterns characteristic of myocardial infarction. In some embodiments, ECG characteristic patterns of the myocardial infarction include the following a) ST elevation or ST depression (as shown in FIG. 11) and b) pathological Q waves. In general, normal rhythm produces four entities—a P wave, a QRS complex, a T wave, and a U wave—each having a fairly unique pattern. For example, the P wave represents atrial depolarization, the QRS complex represents ventricular depolarization, the T wave represents ventricular repolarization, and the U wave represents papillary muscle repolarization.

In some embodiments, the pathological Q waves characteristics typically are exhibited due to a previous myocardial infarction. This is relevant to assessing the current risk as according to the World Health Organization: “Survivors of MI are at increased risk of recurrent infarctions and have an annual death rate of 5%—six times that in people of the same age who do not have coronary heart disease.” See

http://www.who.int/cardiovascular_diseases/priorities/secondary_prevention/country/en/index1.html

In some embodiments, the pathological Q waves characteristics include 1) any Q-wave in ECG leads V2-V3 greater than or equal to (≥) 0.02 seconds or QS complex in ECG leads V2 and V3, 2) Q-wave greater than or equal to (≥) 0.03 seconds and greater than (>) 0.1 mV deep or QS complex in ECG leads I, II, aVL, aVF, or in ECG leads V4-V6 in any two ECG leads of a contiguous lead grouping (in ECG leads I, aVL, V6; V4-V6; II, III, and aVF), and c) R-wave greater than or equal to (≥) 0.04 seconds in ECG leads V1-V2 and R/S≥1 with a concordant positive T-wave in the absence of a conduction defect.

In some embodiments, the myocardial infarction risk assessment subsystem is configured to analyze the respiration information of patient P (obtained from the respiration sensor) to determine a risk of an impending myocardial infraction. For example, myocardial infarction risk assessment subsystem is configured to determine a risk of an impending myocardial infraction by detecting the symptoms of dyspnea. In some embodiments, the myocardial infarction risk assessment subsystem is configured to determine dyspnea onset and dyspnea progression by monitoring 1) the mean inhale-exhale duration, 2) mean respiration rate and/or 3) mean respiration amplitude.

Systematic management of patients suffering high-risk symptoms is essential in emergency medical services. Patients suspected of myocardial infarction presenting with dyspnea have significantly higher short- and long-term mortality than patients with chest pain irrespective of a confirmed myocardial infarction diagnosis. See

https://www.ncbi.nlm.nih.gov/pmc/articles/PMC4751637/

Dyspnea (or shortness of breath) more typically arises as part of the constellation of symptoms in an acute coronary syndrome or myocardial infarction. See

https://www.ncbi.nlm.nih.gov/pmc/articles/PMC5247680/

In some embodiments, dyspnea symptoms are detected using radar sensor 204. In some embodiments, radar sensor 204 is embedded in screen 202. In some embodiments, radar sensor 204 are partially embedded in the transmissive portion of patient carrier 202. In some embodiments, radar sensor 204 are fully embedded in the transmissive portion of patient carrier 202. In some embodiments, system 100 is configured to determine respiration sinusoid signal from the data or information obtained from radar sensor 204. In some embodiments, system 100 is configured to detect respiration distress by scanning the respiration sinusoid signal for dyspnea characteristics. In some embodiments, dyspnea characteristics may include low signal amplitude, increased respiration rate, or both.

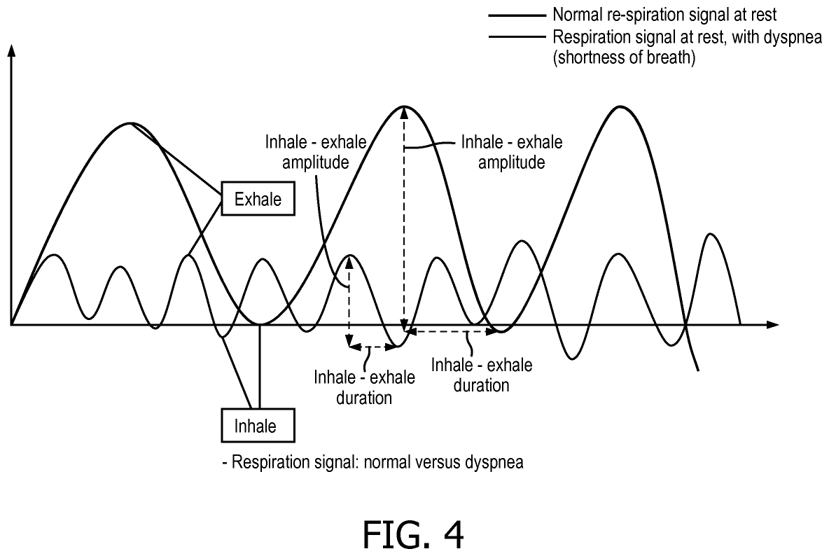

In some embodiments, dyspnea is characterized by shallow and rapid breathing. In some embodiments, shallow breathing is reflected in the respiration signal in the significantly decreased inhale-exhale amplitude compared to normal breathing (as shown in FIG. 4). FIG. 4 shows a graphical representation of the normal respiration signal (at rest) and the respiration signal (at rest) with dyspnea (or shortness of breath). The signal amplitudes of the normal respiration signal and the dyspnea respiration signal (i.e., both signals measured at rest) are shown on the left hand side Y-axis of the graph in FIG. 4 and the time of the normal respiration signal and the dyspnea respiration signal (i.e., both signals measured at rest) are on the X-axis of the graph FIG. 4. The wave with shorter signal amplitude in FIG. 4 represents the dyspnea respiration signal, while the wave with taller signal amplitude in FIG. 4 represents the normal respiration signal. In some embodiments, rapid breathing is reflected in the respiration signal in the high respiration rate (i.e., number of inhale-exhale cycles/min) compared to the normal breathing. Normal respiration rate is typically within 10-18 inhale-exhale cycles/min. Respiration rates at rest (i.e., without emotional or physical exertion) that are higher than 18 cycles/min are outside healthy bounds. A high respiration rate also implies low inhale-exhale duration thereby implying that dyspnea is characterized by a respiratory signal in which the mean inhale-exhale duration is significantly lower than in normal breathing.

In some embodiments, dyspnea onset and progression monitoring is performed by monitoring 1) the mean inhale-exhale duration, 2) mean respiration rate and/or 3) mean respiration amplitude.

In some embodiments, the myocardial infarction risk assessment subsystem is configured to determine myocardial infarction risk score or parameter. In some embodiments, the determined myocardial infarction risk score or parameter is elevated when establishing if the ECG wave exhibits ST elevations. In some embodiments, the myocardial infarction risk parameter is further amplified by the detection of pathological Q waves, dyspnea and finally abnormal heart rate and blood pressure values as below in Equation (1):

MyocardialInfarctionRiskScore=ST_Elevated⊗PathologicalQWaves⊗Dyspnea⊗HeartRateDifftoNorm⊗BloodPressureDifftoNorm Equation (1)

In some embodiments, the myocardial infarction risk assessment subsystem is configured to determine myocardial infarction risk score or parameter using Equation (2) below.

MyocardialInfarctionRiskScore=w1*ST_Elevated+w2*PathologicalQWaves+w3*Dyspnea+w4*HeartRateDifftoNorm+w5* BloodPressureDifftoNorm Equation (2)

In some embodiments, w1, w2, w3, w4 and w5 in equation (2) are weights that are learned/determined from and characteristic to the group of patients similar to the patient at hand.

In some embodiments, the hyperkalemia risk assessment subsystem is configured to monitor and determine the respiratory system status of the patient. In some embodiments, the hyperkalemia risk assessment subsystem is configured to determine the patient hyperkalemia status by automatically analyzing the ECG information obtained from the ECG sensor. In some embodiments, the hyperkalemia status of the patient includes onset/mild hyperkalemia, moderate hyperkalemia, or severe hyperkalemia.

In some embodiments, the hyperkalemia risk assessment subsystem is configured to analyze the ECG information obtained from the ECG sensor to determine whether the ECG signal morphology has changed from its normal characteristics (at normokalemia), towards exhibiting features characteristic of hyperkalemia.

In some embodiments, once characteristic anomalous features are determined, the hyperkalemia risk assessment subsystem is configured to classify the ECG signal pattern to identify the hyperkalemia progression status. The normal ECG signal morphology in comparison with ECG signal features characteristic of the various hyperkalemia phases are presented below.

FIG. 12 shows an example of ECG signal at normokalemia (e.g., for a healthy individual having normal levels of potassium in blood). The illustration in FIG. 12 shows the lesser P and T waves and pronounced QRS peak, with associated intervals and segments in-between (PR, ST and QT). In contrast, medical studies (See “Electrocardiographic manifestations of hyperkalemia,” by Mattu, Amal, William J. Brady, and David A. Robinson in The American journal of emergency medicine 18.6 (2000): 721-729; http://www.ncbi.nlm.nih.gov/pmc/articles/PMC3627796/; “ABC of clinical electrocardiography: Conditions not primarily affecting the heart,” by Slovis C, Jenkins R., in BMJ 2002 Jun. 1; 324(7349):1320-3. DOI: http://dx.doi.org/10.1136/bmj.324.7349.1320 Erratum in: BMJ 2002 Aug. 3; 325(7358):259. BMJ 2007 May 26; 334(7603).

DOI: http://dx.doi.org/10.1136/bmj.39219.615243.AD; “Recognising signs of danger: EKG changes resulting from an abnormal serum potassium concentration,” by Webster A, Brady W, Morris F., in Emerg Med J 2002 Jan. 19; 19(1):74-7. DOI: http://dx.doi. org/10.1136/emj.19.1.74; “Electrocardiographic manifestations: electrolyte abnormalities,” by Diercks D B, Shumaik G M, Harrigan R A, Brady W J, Chan T C., in J Emerg Med 2004 August; 27(2):153-60.
DOI: http://dx.doi.org/10.1016/j.jemermed.2004.04.006; and
http://www.bpac.org.nz/BT/2011/September/imbalance.aspx) show that the ECG signal morphology changes in a specific way according to the various phases of hyperkalemia severity. In that sense in the article (See http://www.ncbi.nlm.nih.gov/pmc/articles/PMC3627796/), “typical ECG in hyperkalemia progress from tall, “peaked” T waves and a shortened QT interval, to lengthening PR interval and loss of P waves, and then to widening of the QRS complex culminating in a “sine wave” morphology and death if not treated” (See FIG. 12).

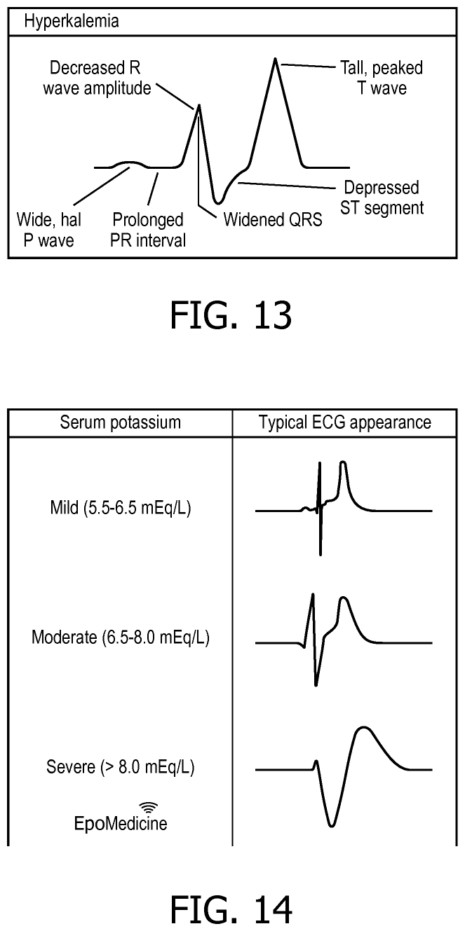

FIG. 13 illustrates this progression, while FIG. 14 shows that ECG final sinusoidal wave at severe stage of hyperkalemia as described in

http://epomedicine.com/emergency-medicine/ecg-changes-hyperkalemia/ FIG. 13 shows ECG QRS pattern at mild, moderate and severe hyperkalemia as depicted in
http://epomedicine.com/emergency-medicine/ecg-changes-hyperkalemia/ In
http://epomedicine.com/emergency-medicine/ecg-changes-hyperkalemia/, the various phases of hyperkalemia are described with corresponding changes in ECG.

In some embodiments, mild hyperkalemia is detected at potassium levels, K in the range between 5.5 and 6.0 milliequivalents of solute per liter of solution (mEq/L), where rapid repolarization causes peaked T waves. In some embodiments, mild hyperkalemia is detected at potassium levels, K in the range between 6.0 and 6.5 mEq/L, where decrease in conduction causes prolonged PR and QT intervals.

In some embodiments, moderate hyperkalemia is detected at potassium levels, K in the range between 6.5 and 7.0 mEq/L, where P waves are diminished and ST segment may be depressed. In some embodiments, moderate hyperkalemia is detected at potassium levels, K in the range between 7.0 and 8.0 mEq/L, where P waves disappear, QRS widens.

In some embodiments, severe hyperkalemia is detected potassium levels, K in the range between 8.0 and 10.0 mEq/L, where QRS merges with T wave to produce classic sine wave (QRS-T fusion—a sinusoidal waveform). In some embodiments, severe hyperkalemia is detected at potassium levels, K in the range between 10.0 and 12.0 mEq/L, where ventricular fibrillation and diastolic arrest occur.

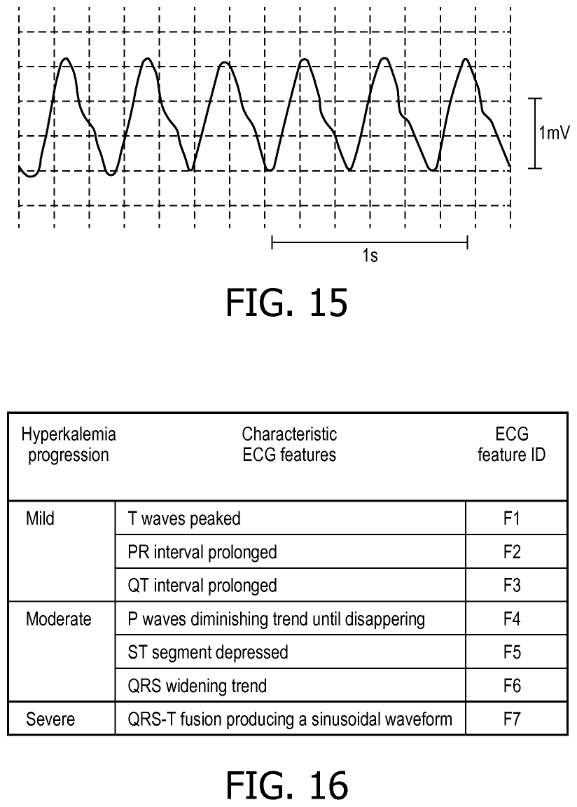

FIG. 15 shows ECG sinusoidal pattern at final stage hyperkalemia as depicted in

https://researchportal.port.ac.uk/portal/files/5878124/thesis_for_Binding_final_copy_.3.pdf

Table in FIG. 16 summarizes the ECG signal features characteristic of each hyperkalemia phase. All ECG features detection pre-require automatic detection of the PQRST wave. For example, for mild hyperkalemia progression, the ECG features are identified as “F1,” “F2” and “F3,” when the corresponding characteristic ECG features include “T waves peaked,” “PR interval prolonged,” and “QT interval prolonged.” For moderate hyperkalemia progression, the ECG features are identified as “F4,” “F5” and “F6,” when the corresponding characteristic ECG features include “P waves diminishing trend until disappearing,” “ST segment depressed,” and “QRS widening trend.” For severe hyperkalemia progression, the ECG feature is identified as “F7,” when the corresponding characteristic ECG features include “QRS-T fusion producing a sinusoidal waveform.”

In some embodiments, an automatic detection of ECG features characteristic of hyperkalemia progression using the hyperkalemia risk assessment subsystem, include the following procedures. The first procedure is an automatic detection of the PQRST wave—done by applying real time signal processing peak detection techniques to identify P, Q, R, S, and T points, as well as l₁, l₂, l₃, and l₄ (as depicted in FIG. 12). This allows identifying their corresponding amplitudes and time stamps throughout the ECG signal. At the next procedure, P, Q, R, S, T points amplitudes (Amp) and time stamps (TS) are stored in associated vectors, including, P_Amp, P_TS, Q_Amp, Q_TS, R_Amp, R_TS, S_Amp, S_TS, T_Amp, T_TS, l₁_Amp, l₂_Amp, l₃_Amp, l₄_Amp, l₁_TS, l₂_TS, l₃_TS, and l₄_TS.

In some embodiments, for mild hyperkalemia progression, the ECG feature identified as “F1,” and the corresponding characteristic ECG features include “T waves peaked,” the automatic detection of ECG features characteristic of hyperkalemia progression using the hyperkalemia risk assessment subsystem includes the procedures of trend detection within the T_Amp vector in order to determine increasing amplitude of the T wave (peaking). In some embodiments, the hyperkalemia risk assessment subsystem is configured to confirm the presence of F1 when 1) significant increasing trend is established in T_Amp and 2) there are instances of T_Amp>=R_Amp (i.e., within the same PQRST wave).

In some embodiments, for mild hyperkalemia progression, the ECG feature identified as “F2,” and the corresponding characteristic ECG features include “PR interval prolonged,” the automatic detection of ECG features characteristic of hyperkalemia progression using the hyperkalemia risk assessment subsystem includes the procedures of 1) calculating the PR interval within each PQRST wave based on the corresponding P_TS and R_TS values; and b) trend detection over the PR intervals length. In some embodiments, the hyperkalemia risk assessment subsystem is configured to confirm the presence of F2 when a significantly increasing trend of values within the PR intervals is determined.

In some embodiments, for mild hyperkalemia progression, the ECG feature identified as “F3,” and the corresponding characteristic ECG features include “QT interval prolonged,” the automatic detection of ECG features characteristic of hyperkalemia progression using the hyperkalemia risk assessment subsystem includes the procedures of 1) calculating the QT interval length within each PQRST wave based on the corresponding Q_TS and T_TS values and b) trend detection over the QT intervals length. In some embodiments, the hyperkalemia risk assessment subsystem is configured to confirm the presence of F3 when a significantly increasing trend of values within the QT intervals length is determined.

In some embodiments, for moderate hyperkalemia progression, the ECG feature identified as “F4,” and the corresponding characteristic ECG features include “P waves diminishing trend until disappearing,” the automatic detection of ECG features characteristic of hyperkalemia progression using the hyperkalemia risk assessment subsystem includes the procedures of trend detection within the P_Amp vector in order to determine decreasing amplitude of the P wave. In some embodiments, the hyperkalemia risk assessment subsystem is configured to confirm the presence of F4 when 1) significant decreasing trend is established in the P_Amp vector and 2) P_Amp values approach 0 flat values within ε.

In some embodiments, for moderate hyperkalemia progression, the ECG feature identified as “F5,” and the corresponding characteristic ECG features include “ST segment depressed,” the automatic detection of ECG features characteristic of hyperkalemia progression using the hyperkalemia risk assessment subsystem includes the procedures of 1) determining ST segment depression trend and 2) determining ST segment length decreasing trend.

In some embodiments, the procedure of determining ST segment depression trend includes calculating the ST segment orientation by calculating the difference D_Amp=l₄_Amp−l₃_Amp. In normal ST segments, D_Amp is close to 0 (i.e., ST segment is close to horizontal). In contrast, in depressed ST segments the value of this difference is significantly higher. In some embodiments, the procedure of determining ST segment depression trend also includes trend detection over D_Amp values over time to determine ST segment depression trend.

In some embodiments, the procedure of determining ST segment length decreasing trend includes 1) calculating the ST segment length within each PQRST wave based on the corresponding D_TS=l₃_TS and l₄_TS values, and 2) trend detection over D_TS values over time to determine ST segment length decrease.

In some embodiments, the hyperkalemia risk assessment subsystem is configured to confirm the presence of F5 when at least one of the trends above is established.

In some embodiments, for moderate hyperkalemia progression, the ECG feature identified as “F6,” and the corresponding characteristic ECG features include “QRS widening trend,” the automatic detection of ECG features characteristic of hyperkalemia progression using the hyperkalemia risk assessment subsystem includes the procedures of 1) calculating the length of each QRS complex based on the corresponding Q_TS and S_TS values; and 2) trend detection over the QRS complex lengths. In some embodiments, the hyperkalemia risk assessment subsystem is configured to confirm the presence of F6 when a significantly increasing trend of values within the QRS complex length is determined.

In some embodiments, for severe hyperkalemia progression, the ECG feature identified as “F7,” and the corresponding characteristic ECG features include “QRS-T fusion producing a sinusoidal waveform,” the automatic detection of ECG features characteristic of hyperkalemia progression using the hyperkalemia risk assessment subsystem. In some embodiments, the hyperkalemia risk assessment subsystem is configured to confirm the presence of F7 when 1) F1-F6 are detected and 2) sinusoid detected by applying pattern recognition techniques.

In some embodiments, the automatic assessment of hyperkalemia risk score includes calculation of hyperkalemia risk score based on the detection of features above resulting in three levels of risk: mild hyperkalemia, moderate hyperkalemia, and severe hyperkalemia. In some embodiments, the hyperkalemia risk assessment subsystem is configured to confirm the onset of mild hyperkalemia when features F1, F2, and F3 are detected over a defined period of time (epoch). In some embodiments, the hyperkalemia risk assessment subsystem is configured to confirm the onset of moderate hyperkalemia when features F4, F5, and F6 are detected over a defined period of time (epoch). In some embodiments, the hyperkalemia risk assessment subsystem is configured to confirm the onset of severe hyperkalemia when feature F7 are detected over a defined period of time (epoch).

In some embodiments, hypokalemia risk assessment subsystem is configured to monitor and determine the respiratory system status of the patient. In some embodiments, hypokalemia risk assessment subsystem is configured to determine the patient hypokalemia status (i.e., onset/mild, moderate, or severe) by automatically analyzing the ECG input signal to determine whether the ECG signal morphology has changed from its normal characteristics (i.e., at normokalemia), towards exhibiting features characteristic of hypokalemia. In some embodiments, once characteristic anomalous features are determined, the hypokalemia risk assessment subsystem is configured to classify the signal pattern to identify the hypokalemia progression status.

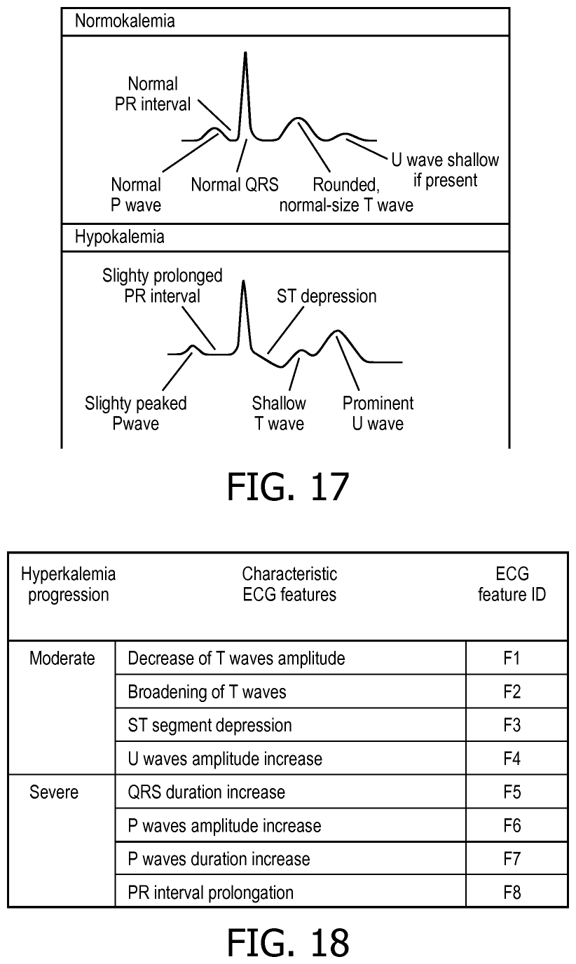

The normal ECG signal morphology in comparison with ECG signal features characteristic of the various hypokalemia phases are presented below. FIG. 17 illustrates an example of ECG signal at normokalemia (i.e., healthy individual, normal levels of potassium in blood). The illustration shows the lesser P and T waves and pronounced QRS peak, with associated intervals and segments in-between (PR, ST and QT). FIG. 17 also illustrates a comparison between normal ECG wave at normokalemia (i.e., healthy individual, normal levels of potassium in blood) and ECG wave changes at hypokalemia.

In contrast, medical studies show that the ECG signal morphology changes in specific way according to the various phases of hypokalemia severity. In that sense Diercks et al. (See “Electrocardiographic manifestations: electrolyte abnormalities,” by Diercks D B, Shumaik G M, Harrigan R A, Brady W J, Chan T C. in J Emerg Med 2004 August; 27(2):153-60. DOI: http://dx.doi. org/10.1016/j.jemermed.2004.04.006) found that hypokalemia causes first a decrease in the T wave amplitude, followed by ST segment depression and actual T wave inversions in correspondence to a further decrease in potassium level. Moreover, PR interval increases and P wave amplitude can increase as well. Severe hypokalemia manifests a prominent U wave, a positive deflection after the T-wave.

The classification for hypokalemia based on potassium, K level was proposed by the medical study “Prevalence of severe hypokalaemia in patients with traumatic brain injury. Injury,” by Wu X, Lu X, Lu X, Yu J, Sun Y, Du Z, Wu X, Mao Y, Zhou L, Wu S, Hu J. in January 2015; 46(1):35-41. doi: 10.1016/j.injury.2014.08.002. Epub 2014 Aug. 10. According to this classification, when the potassium levels are between 3.0 mmol/L (a molar concentration, measured in millimoles per litre) and 3.5 mmol/L, hypokalemia is classified as mild hypokalemia. When the potassium levels are between 2.5 mmol/L and 3.0 mmol/L, hypokalemia is classified as moderate hypokalemia. When the potassium levels are less than 2.5 mmol/L, hypokalemia is classified as severe hypokalemia.

In some embodiments, the above-noted medical studies and the medical study “Electrolyte disorders and arrythmogenesis,” by El-Sherif N, Turitto G. in Cardiol J 2011; 18(3):233-45 lead to the following features characterization: 1) moderate hypokalemia shows decrease in amplitude and broadening of T waves, ST segment depression and increase of U wave amplitude; and 2) severe hypokalemia shows an increase of QRS duration (without a concomitant change in the QRS configuration), increase in P wave amplitude and duration and a prolongation of P-R interval.

Table in FIG. 18 summarizes the ECG signal features characteristic of each hypokalemia phase. In some embodiments, for moderate hypokalemia progression, the ECG feature is identified as “F1,” “F2” “F3,” and “F4,” when the corresponding characteristic ECG features include “decrease of T waves amplitude,” “broadening of T waves,” “ST segment depression,” and “U waves amplitude increase.” For severe hypokalemia progression, the ECG feature is identified as “F5,” “F6,” “F7,” and “F8,” when the corresponding characteristic ECG features include “QRS duration increase,” “P waves amplitude increase,” “P waves duration increase,” and “PR interval prolongation.”

Automatic detection of ECG features characteristic of the hypokalemia progression are discussed here. In some embodiments, automatic detection of the PQRST(U) wave is performed by applying real time signal processing peak detection techniques to identify P, Q, R, S, T, and (U) points, as well as l₀, l₁, l₂, l₃, l₄, and l₅. This will allow identifying their corresponding amplitudes and time stamps throughout the ECG signal. The procedure of detecting the ECG signal features identifying hypokalemia progression is similar as for hyperkalemia above and, therefore, will not be described in great detail here.

In some embodiments, the automatic assessment of hypokalemia risk score includes calculation of hypokalemia risk score based on the detection of features above resulting in two levels of risk: moderate hypokalemia and severe hypokalemia. In some embodiments, the hypokalemia risk assessment subsystem is configured to confirm the onset of moderate hypokalemia when features F1, F2, F3, and F4 are detected over a defined period of time (epoch). In some embodiments, the hypokalemia risk assessment subsystem is configured to confirm the onset of severe hypokalemia when features F5, F6, F7, and F8 are detected over a defined period of time (epoch).

In some embodiments, cardiovascular system analysis subsystem 114 is configured to monitor and determine the cardiovascular system status of the patient. In some embodiments, cardiovascular system analysis subsystem 114 is configured to determine the cardiovascular distress as described below.

In some embodiments, cardiovascular system analysis subsystem 114 is configured to determine cardiovascular risk score using the wearable ECG sensor and blood pressure sensors applied to patient wrist, allowing measurement of heart rate, along with the analysis of the PQRST complex. In some embodiments, cardiovascular system analysis subsystem 114 is configured to determine cardiovascular distress based on: 1) myocardial infarction risk assessment by the myocardial infarction risk assessment subsystem; 2) hyperkalemia risk assessment by the hyperkalemia risk assessment subsystem; and 3) hypokalemia risk assessment by the hypokalemia risk assessment subsystem. In some embodiments, the cardiovascular risk score is a vector comprising the three components above.

In some embodiments, based on the values of each of the three component risk scores (i.e., myocardial infarction risk score, hypokalemia risk assessment score, hyperkalemia risk assessment score), system 100 (including cardiovascular system analysis subsystem 114) is configured to 1) assesses the overall patient condition severity and emergency risk, 2) calculate projections regarding the patient expected deterioration rate in case no intervention is provided based on similar cases and patients; 3) advice optimal intervention timeframe and medical specialists needed for the case; 4) update the triage list; 4) inform medical staff of a) patient current status and deterioration projection, b) optimal intervention time, c) update to triage list.

In some embodiments, emergency risk assessment subsystem 120 is configured to assess the emergency risk of a patient based on input provided by all subsystems above and assist in patient triage at the emergency department. In one embodiment, the emergency risk parameter is a combination of the risk scores put out by the all the subsystems discussed above, where each individual risk score/parameter can be given a particular weight that can be adapted based on the patient clinical profile. In addition, the emergency risk parameter is initialized with an inherent risk score/parameter calculated based on the patient clinical profile—i.e., patient age, known conditions etc. In some embodiments, emergency risk assessment subsystem 120 is configured to assess the emergency risk of a patient based on input provided by 1) respiratory system analysis subsystem 112; 2) cardiovascular system analysis subsystem 114; 3) stroke risk assessment subsystem 116; and 4) fracture scan and detection subsystem 118 and assist in patient triage at the emergency department. In some embodiments, emergency risk assessment subsystem 120 is configured to assess the emergency risk of a patient based on input provided by 1) respiratory system analysis subsystem 112; 2) cardiovascular system analysis subsystem 114; and 3) fracture scan and detection subsystem 118 and assist in patient triage at the emergency department. In some embodiments, emergency risk assessment subsystem 120 is configured to assess the emergency risk of a patient based on input provided by 1) respiratory system analysis subsystem 112; 2) cardiovascular system analysis subsystem 114; 3) stroke risk assessment subsystem 116; and 4) injury scan and detection subsystem 124 and assist in patient triage at the emergency department. In some embodiments, emergency risk assessment subsystem 120 is configured to assess the emergency risk of a patient based on input provided by 1) respiratory system analysis subsystem 112; 2) cardiovascular system analysis subsystem 114; and 3) injury scan and detection subsystem 124 and assist in patient triage at the emergency department. In some embodiments, emergency risk assessment subsystem 120 is configured to assess the emergency risk of a patient based on input provided by 1) respiratory system analysis subsystem 112; 2) cardiovascular system analysis subsystem 114; 3) stroke risk assessment subsystem 116; 4) fracture scan and detection subsystem 118; 5) body contour detection subsystem 122; and 6) injury scan and detection subsystem 124 and assist in patient triage at the emergency department.

In some embodiments, one or more sensors 106a . . . 106n; 107a . . . 107n are operatively associated with the patient and/or operatively connected with patient support 202 configured to support patient P. In some embodiments, one or more sensors 107a . . . 107n include a blood pressure sensor for measuring the blood pressure information of the patient, an Electrocardiography (ECG) sensor for measuring the Electrocardiography (ECG) information of the patient, and a heart rate sensor for measuring the heart rate information of the patient. In some embodiments, one or more sensors 107a . . . 107n include an audio sensor for obtaining the injury information of the patient, a thermal sensor (for obtaining thermal infrared images) for obtaining the injury/fracture information of the patient and/or for obtaining the body contour information of the patient; and a RGB sensor for obtaining visible images for obtaining the injury/fracture information of the patient and/or for obtaining the respiratory information of the patient. In some embodiments, one or more sensors 106a . . . 106n, include a respiration sensor for measuring the respiration information of the patient. Each of these sensors 106a . . . 106n; 107a . . . 107n are described in detail in the discussion below.

In some embodiments, one or more sensors 106a . . . 106n; 107a . . . 107n may include sensor systems and/or other medical devices that are configured for monitoring patient health information, such as heart rate, ECG waves, respiration waves, blood pressure, breathing or respiration rate, etc.

In some embodiments, one or more sensors 106a . . . 106n include optical or radar sensors. In some embodiments, optical or radar sensors 106a . . . 106n are embedded in the transmissive structure of patient carrier 202. In some embodiments, one or more sensors 106a . . . 106n are configured to measure respiration information of the patient via light or radio waves traveling through at least a portion of the transmissive structure of patient carrier 202. In some embodiments, one or more optical or radar sensors 106a . . . 106n comprises one or more radar sensors (i) embedded in the transmissive portion of the patient carrier and (ii) configured to measure the respiration information of the patient via radio waves traveling through at least a portion of the transmissive structure of patient carrier 202. In some embodiments, one or more optical or radar sensors 106a . . . 106n comprises one or more optical sensors (i) embedded in the transmissive portion of the patient carrier and (ii) configured to measure the respiration information of the patient via light traveling through at least a portion of the transmissive structure of patient carrier 202.

In some embodiments, one or more optical sensors 106a . . . 106n are partially embedded in the transmissive portion of patient carrier 202. In some embodiments, one or more optical sensors 106a . . . 106n are fully embedded in the transmissive portion of patient carrier 202. In some embodiments, one or more radar sensors 106a . . . 106n are partially embedded in the transmissive portion of patient carrier 202. In some embodiments, one or more radar sensors 106a . . . 106n are fully embedded in the transmissive portion of patient carrier 202.

In some embodiments, one or more sensors 107a . . . 107n include a heart rate sensor for measuring the heart rate information of patient P. In some embodiments, the heart rate sensor is implemented in a wrist device (like a smart watch) or other wearable devices, i.e., devices that can be attached or arranged on the skin of patient P. In some embodiments, the heart rate sensor can be an optical sensor for determining the heart rate data of patient P. In some embodiments, the heart rate sensor is a non-invasive heart rate sensor that is able to measure the electrical, acoustical or optical activity of the heart or the cardio-respiratory system. In some embodiments, the heart rate sensor is configured to communicate wirelessly with computer system 102. In some embodiments, the wearable heart rate sensor is applied to patient's wrist, allowing measurement of heart rate, along with the analysis of the PQRST complex. In some embodiments, the heart rate sensor includes an electrode operatively associated with patient P and to measure the heart rate information of patient P, and a transmitter for sending the heart rate information to computer system 102.

In some embodiments, one or more sensors 106a . . . 106n include a radar sensor 204 (as shown in FIG. 2) for measuring respiration information of patient P. In some embodiments, the radar sensor includes a transmitter for sending signals to patient P and a receiver for receiving the signals from patient P. In some embodiments, the radar sensor is partially or fully embedded in patient support 202. In some embodiments, the radar sensor is configured to communicate wirelessly with computer system 102. In some embodiments, one or more sensors 106a . . . 106n include other respiration sensor(s) for measuring respiration information of patient P. In some embodiments, the respiration sensor is configured to communicate wirelessly with computer system 102. In some embodiments, the respiration sensor is implemented as a wearable device, i.e., device that can be attached or arranged on the skin of patient P.

In some embodiments, one or more sensors 107a . . . 107n include a electrocardiogram (ECG) sensor for measuring Electrocardiography (ECG) information of patient P. In some embodiments, the electrocardiogram (ECG) sensor is implemented as a wearable device, i.e., device that can be attached or arranged on the skin of patient P. In some embodiments, the wearable electrocardiogram (ECG) sensor is applied to patient's wrist, allowing measurement of heart rate, along with the analysis of the PQRST complex. In some embodiments, the electrocardiogram (ECG) sensor is configured to communicate wirelessly with computer system 102. In some embodiments, the electrocardiogram (ECG) sensor may also be referred to as wearable EKG sensor. In some embodiments, the electrocardiogram (ECG) sensor is configured to record the electrical activity of the heart over a period of time using electrodes placed on the patient's skin. In some embodiments, the electrocardiogram (ECG) sensor includes an electrode operatively associated with patient P and to measure the ECG information of patient P, and a transmitter for sending the ECG information to computer system 102.

In some embodiments, one or more sensors 107a . . . 107n include a blood pressure sensor for measuring blood pressure information of patient P. In some embodiments, the blood pressure sensor is implemented as a wearable device, i.e., device that can be attached or arranged on the skin of patient P. In some embodiments, the blood pressure sensor is configured to communicate wirelessly with computer system 102.

In some embodiments, one or more sensors 107a . . . 107n include an audio/a sound sensor 208/214. For example, in some embodiments, audio sensor comprises a sound sensor 208. In some embodiments, the audio sensor comprises a stethoscope 214. In some embodiments, the audio sensor comprises sound sensor 208 and/or stethoscope 214. In some embodiments, the audio sensor includes a (sound signal) transmitter for sending signals to patient P and a (sound signal) receiver for receiving the signals from patient P.

In some embodiments, one or more sensors 107a . . . 107n include a thermal sensor/detector 210. For example, in some embodiments, the thermal sensor comprises an Infrared camera 210. In some embodiments, the thermal camera is configured to identify injuries/fractures. In some embodiments, the thermal sensor is configured to measure heat by changes in heat and infrared energy. In some embodiments, the thermal sensor is configured to capture thermal infrared images of the patient. In some embodiments, the thermal sensor may include a processor configured to process the captured thermal infrared image data and to transmit the processed thermal infrared image data to subsystems 112-124 and 129. In some embodiments, the thermal sensor may transmit the captured thermal infrared image data to subsystems 112-124 and 129 without processing.

In some embodiments, one or more sensors 107a . . . 107n include a multispectral camera. In some embodiments, the multiple spectral camera includes a five wavelengths camera with a wide spectrum light source. In some embodiments, the multispectral camera is configured to measure the SPO₂ levels. In some embodiments, the multispectral camera may include a processor configured to process the captured image data and to transmit the processed image data to subsystems 112-124 and 129. In some embodiments, the multispectral camera may transmit the captured image data to subsystems 112-124 and 129 without processing.

In some embodiments, one or more sensors 107a . . . 107n include an image sensor 212/206. For example, in some embodiments, the image sensor comprises a RGB camera 212/206. In some embodiments, the image sensor is configured to capture visible light images of the patient. In some embodiments, the RGB camera uses a wide spectrum light source. In some embodiments, the RGB camera uses ambient light source. In some embodiments, the image sensor/RGB camera is configured to measure SPO₂ levels. In some embodiments, the image sensor/RGB camera is configured to distinguish between internal and external injuries. In some embodiments, the image sensor may include a processor configured to process the captured image data and to transmit the processed image data to subsystems 112-124 and 129. In some embodiments, the image sensor may transmit the captured image data to subsystems 112-124 and 129 without processing.

In some embodiments, the blood pressure information of the patient, the Electrocardiography (ECG) information of the patient and/or the heart rate information of the patient may together be referred to as cardiac information of the patient. In some embodiments, the heart rate sensor and the ECG sensor may be integrally formed as a single sensor.

In some embodiments, the cardiac information, the injury information and the respiration information may be obtained from a database 132 that is being updated in real-time by one or more sensors 106a . . . 106n; 107a . . . 107n.

In one scenario, one or more sensors 106a . . . 106n; 107a . . . 107n may provide the cardiac information, the injury information and the respiration information to a computer system (e.g., comprising server 102) over a network (e.g., network 150) for processing. In another scenario, upon obtaining the cardiac information, the injury information and the respiration information, the sensors may process the obtained cardiac information, the obtained injury information and the obtained respiration information, and provide processed cardiac information, processed injury information and processed respiration information to the computer system (e.g., comprising server 102) over a network (e.g., network 150).

In some embodiments, referring to FIG. 2, system 100 includes the screen or patient support 202 that is equipped with a number of sensors 106a . . . 106n; 107a . . . 107n including 204, 206, 208, 210, 212, and 214 that are used to scan patient P, assess his condition severity and emergency risk, which is used in updating the triage list in the Emergency Department. In some embodiments, system 100 includes a carrier support or member 216 operatively connected to patient support 202. In some embodiments, sound emitter 208 and stethoscope 214 are attached to carrier support 216.

In some embodiments, patient P is placed or positioned on screen 202. In some embodiments, patient P reclines against screen 202 (i.e., in an oblique position angle) with hand palms placed against screen 202, arms away from the body and legs at a distance from one another.

In some embodiments, screen 202 is made of transparent plastic material.

In some embodiments, screen 202 includes IR/thermal camera 210 and RGB camera 212 both positioned above screen 202. In some embodiments, RGB camera 206 positioned below screen 202 such that patient P can be scanned from above and below (i.e., front and back). In some embodiments, screen 202 embeds a matrix of optical sensors 217 in its mid-section where the patient palms are expected to be placed. In some embodiments, radar sensor 204 is embedded with screen 202. In some embodiments, radar sensor 204 is placed beneath screen 202. In some embodiments, the transmissive structure can be made of, including glass material, plastic material, etc. In some embodiments, radio and/or optical waves can travel easily through transmissive structure. In some embodiments, “transmissive” may be transparent in the context of light, but otherwise means a material through which information/data can pass through. In some embodiments, it can be opaque in the case of radio wave embodiments.

In some embodiments, system 100 is configured to automatically determine the patient body contour. In some embodiments, system 100 is configured to screen patient P. In some embodiments, system 100 is configured to assess the probability for fracture severity. In some embodiments, system 100 is configured to assess the probability for injury severity. In some embodiments, system 100 is configured to assess the overall patient condition severity and emergency risk. In some embodiments, system 100 is configured to determine the cardiovascular emergency risk. In some embodiments, system 100 is configured to assess the overall patient condition severity and emergency risk. In some embodiments, system 100 is configured to calculate projections regarding the patient expected deterioration rate in case no medical intervention is provided based on similar cases and patients. In some embodiments, system 100 is configured to advise optimal intervention timeframe and medical specialists needed for the case. In some embodiments, system 100 is configured to update the triage list. In some embodiments, system 100 is configured to inform medical staff of a) patient current status and deterioration projection, b) optimal intervention time, and c) update to the triage list in the Emergency Department.

In some embodiments, body contour detection subsystem 122 is configured to detect the contour of the patient body. In some embodiments, the contour of the patient body includes the position of head, limbs and palms. In some embodiments, body contour detection subsystem 122 is configured to detect the body contour using InfraRed (IR) camera 210. In some embodiments, IR camera 210 is mounted on a support 215 that moves along the length of the patient body allowing IR camera 210 to scan the whole body of the patient and determine its position as well as the position of patient P. In some embodiments, IR camera 210 is configured to move along the length of the patient body allowing IR camera 210 to scan 1) the head of the patient and determine its position; 2) the arms of the patient and determine their positions; 3) the hands of the patient and determine their positions; 4) the legs of the patient and determine their positions; and/or 5) the body trunk of the patient and determine its position.

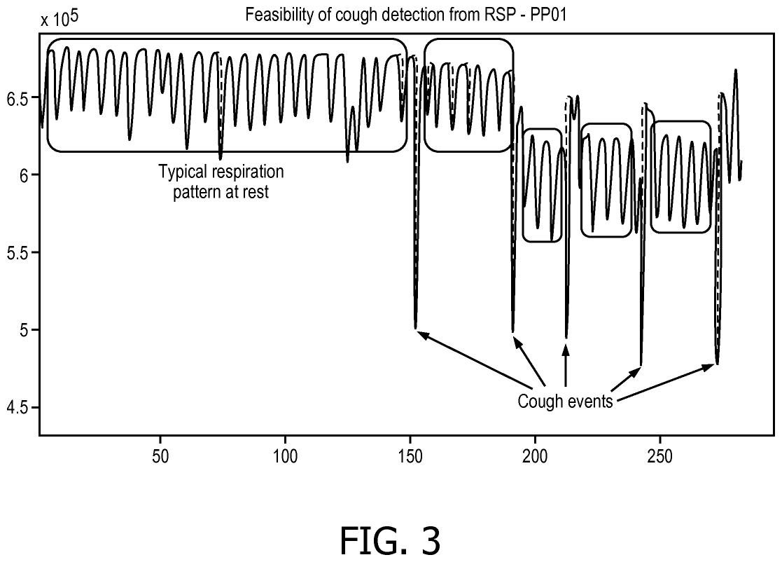

In some embodiments, respiratory system analysis subsystem 112 is configured to monitor and determine the status of the respiratory system of the patient. In some embodiments, respiratory system analysis subsystem 112 is configured to provide respiratory system risk assessment parameter to patients coming to the Emergency Department. That is, system 100 provides an automatic solution for the assessment of respiratory risk of patients coming to the Emergency Department. In some embodiments, respiratory system analysis subsystem 112 is configured to detect respiratory distress by scanning the patient to determine 1) respiratory depression; 2) coughing events; 3) dyspnea symptoms; 4) peripheral cyanosis; 5) SPO₂ level; and/or 6) fever. In some embodiments, the respiratory depression is characterized by low frequency, low amplitude respiratory cycles. In some embodiments, the coughing events are detected by executing peak detection and comparison of the difference between the signal amplitude values of each adjacent high peak vs. low peak in the respiration signal. In some embodiments, as shown in FIG. 3, if the difference between the value of each two (i.e., high vs. low) adjacent peaks is higher than a threshold, then the low peak indicates the time stamp of the cough event. FIG. 3 illustrates the feasibility of this approach.

Systematic management of patients suffering high-risk symptoms is essential in emergency medical services. Patients suspected of myocardial infarction presenting with dyspnea have significantly higher short- and long-term mortality than patients with chest pain irrespective of a confirmed myocardial infarction diagnosis. See

https://www.ncbi.nlm.nih.gov/pmc/articles/PMC4751637/

Dyspnea (or shortness of breath) more typically arises as part of the constellation of symptoms in an acute coronary syndrome or myocardial infarction. See https://www.ncbi.nlm.nih.gov/pmc/articles/PMC5247680/ In some embodiments, respiratory system analysis subsystem 112 is configured to determine the status of the respiratory system of the patient by detecting the symptoms of dyspnea.

In some embodiments, the dyspnea symptoms are detected using radar sensor 204. In some embodiments, radar sensor 204 is embedded in screen 202. In some embodiments, radar sensor 204 is partially embedded in screen 202. In some embodiments, radar sensor 204 is fully embedded in screen 202. In some embodiments, system 100 is configured to determine respiration sinusoid signal from the data or information obtained from radar sensor 204. In some embodiments, system 100 is configured to detect respiration distress by scanning the respiration sinusoid signal for dyspnea characteristics. In some embodiments, dyspnea characteristics may include low signal amplitude, increased respiration rate, or both. In some embodiments, the dyspnea information is obtained from the respiration sensor, respectively.

In some embodiments, dyspnea is characterized by shallow and rapid breathing. In some embodiments, shallow breathing is reflected in the respiration signal in the significantly decreased inhale-exhale amplitude compared to normal breathing (as shown in FIG. 4). FIG. 4 shows a graphical representation of the normal respiration signal (at rest) and the respiration signal (at rest) with dyspnea (or shortness of breath). The signal amplitudes of the normal respiration signal and the dyspnea respiration signal (i.e., both signals measured at rest) are shown on the left hand side Y-axis of the graph in FIG. 4 and the time of the normal respiration signal and the dyspnea respiration signal (i.e., both signals measured at rest) are on the X-axis of the graph FIG. 4. The wave with shorter signal amplitude in FIG. 4 represents the dyspnea respiration signal, while the wave with taller signal amplitude in FIG. 4 represents the normal respiration signal. In some embodiments, rapid breathing is reflected in the respiration signal in the high respiration rate (i.e., number of inhale-exhale cycles/min) compared to the normal breathing. Normal respiration rate is typically within 10-18 inhale-exhale cycles/min. Respiration rates at rest (i.e., without emotional or physical exertion) that are higher than 18 cycles/min are outside healthy bounds. A high respiration rate also implies low inhale-exhale duration thereby implying that dyspnea is characterized by a respiratory signal in which the mean inhale-exhale duration is significantly lower than in normal breathing.

In some embodiments, dyspnea onset and progression monitoring is performed by monitoring 1) the mean inhale-exhale duration, 2) mean respiration rate and/or 3) mean respiration amplitude.

In some embodiments, respiratory system analysis subsystem 112 is configured to determine the status of the respiratory system of the patient by detecting the peripheral cyanosis. In some embodiments, optical sensors 217 are used to detect the peripheral cyanosis. In some embodiments, optical sensors 217 are embedded in screen or carrier 202. In some embodiments, optical sensors 217 are partially embedded in screen or carrier 202. In some embodiments, optical sensors 217 are fully embedded in screen or carrier 202. In some embodiments, optical sensors 217 are embedded in the hands profiles that are part of screen or carrier 202. In some embodiments, optical sensors 217 are configured to scan the hands of the patient for blue spectrum coloration of the epidermis indicating lack of oxygen in peripheral tissues associated with advanced respiratory distress. In some embodiments, if coloration is established, respiratory system analysis subsystem 112 of system 100 indicates the detection of the peripheral cyanosis.

In some embodiments, system 100 includes peripheral cyanosis detection subsystem that is configured to determine the peripheral cyanosis status of the patient. In some embodiments, respiratory system analysis subsystem 112 includes peripheral cyanosis detection subsystem that is configured to determine the peripheral cyanosis status of the patient.

In some embodiments, the peripheral cyanosis detection subsystem assumes that the patient hands have been using vision techniques recognize various parts of the hand (i.e., center/palm vs fingers/extremities).

In some embodiments, the peripheral cyanosis detection is based on the detection of SPO₂ levels in the hands peripheries (i.e., fingers) and by comparing it with the SPO₂ levels in other parts of the patient body where normal SPO₂ levels still hold.

In some embodiments, system 100 uses a SPO₂ level measuring method as described below. In some embodiments, system 100 is configured to receive input from the RGB camera while using ambient light as light source. In some embodiments, the system 100 is configured to use 1) an RGB camera with a wide spectrum light source; and 2) a multispectral camera (e.g., 5 wavelengths) with a wide spectrum light source. The RBG camera and/or the multispectral camera used for detection of the peripheral cyanosis are described in detail in the discussions above.

In some embodiments, respiratory system analysis subsystem 112 is configured to determine the status of the respiratory system of the patient by monitoring the SPO₂ level. The human body requires and regulates a very precise and specific balance of oxygen in the blood. Normal blood oxygen levels in humans are considered to be at 95-100 percent. Blood oxygen levels below 80 percent may compromise organ function, such as the brain and heart. Continued low oxygen levels may lead to respiratory arrest and/or or cardiac arrest. In some embodiments, respiratory system analysis subsystem 112 of system 100 uses a sensor (e.g., two sources of light and one photodiode) to determine the SPO₂ level.

In some embodiments, respiratory system analysis subsystem 112 is configured to determine the status of the respiratory system of the patient by monitoring the fever of the patient. In some embodiments, the fever of the patient is measured using thermal/IR camera 210. In some embodiments, the fever of the patient is measured using thermal/IR camera 210, based on the data regarding the patient head position detected by the body contour detection subsystem 122.

In some embodiments, respiratory system analysis subsystem 112 is configured to determine a respiratory system risk parameter based on the 1) respiratory depression; 2) coughing events; 3) dyspnea symptoms; 4) peripheral cyanosis; 5) SPO₂ level; and/or 6) fever. In some embodiments, the respiratory system risk parameter is increased with each symptom detected above (including, but not limited to, 1) respiratory depression; 2) coughing events; 3) dyspnea symptoms; 4) peripheral cyanosis; 5) SPO₂ level; and/or 6) fever). In some embodiments, the respiratory system risk parameter is configured to escalate from coughing symptoms to fever, to dyspnea, to peripheral cyanosis, and to unbalanced SPO₂ level.

In some embodiments, respiratory system analysis subsystem 112 is configured to determine a respiratory system risk parameter based on the determined/detected peripheral cyanosis. In some embodiments, system 100 is configured to measure reflected light at different wavelengths (e.g., 680 nanometers (nm), 810 nm, 960 nm, etc.) both at the reference location (i.e., a location where normal SPO₂ levels still hold) and the target location. In some embodiments, the ratio of reflected light at the different wavelengths is related to the level of oxygen in blood. In some embodiments, a level of SPO₂ at the reference location and a level of SPO₂ at the target location (e.g., lips) are received by a peripheral cyanosis detection subsystem. In some embodiments, the measurements of the level of SPO₂ at the reference location and the level of SPO₂ at the target location (e.g., lips) are compared. In some embodiments, the measurements of the level of SPO₂ at the reference location and the level of SPO₂ at the target location (e.g., lips) are taken spatially. In some embodiments, a baseline level of SPO₂ (i.e., middle of palm or arm) are compared with a level of SPO₂ at extremities (i.e., same for forehead vs lips). In some embodiments, the difference between the level of SPO₂ at the reference location and the level of SPO₂ at the target location (e.g., lips) are evaluated by the peripheral cyanosis detection system. In some embodiments, the peripheral cyanosis detection subsystem is optional. In some embodiments, the higher the difference between the level of SPO₂ at the reference location and the level of SPO₂ at the target location (e.g., lips), the more acute condition, or more chance of peripheral cyanosis.

In some embodiments, system 100 is also configured to measure CO₂ levels by using the same technique (i.e., the peripheral cyanosis detection method) as described above but with appropriate wavelengths corresponding to the absorption lines of CO₂. Excessive CO₂ levels may lead to hypercapnia. In some embodiments, this peripheral cyanosis detection technique provides an innovative method for capnography during the intensive care, for the triage at the emergency unit.

In some embodiments, injury scan and detection subsystem 124 is configured to automatically detect injuries. In some embodiments, injury scan and detection subsystem 124 is configured to automatically detect fractures and wounds. In some embodiments, system 100 is configured to 1) determine body contour of the patient using body contour detection subsystem 122; and 2) identify injuries. In some embodiments, system 100 is configured to distinguish internal injuries from external injuries.

In some embodiments, system 100 is configured to scan the patient body using thermal/IR camera(s) 210. In some embodiments, system 100 is configured to scan the patient body using thermal/IR camera(s) 210 from top and below. In some embodiments, system 100 uses thermography techniques to identify injuries (i.e., internal or external) as described in

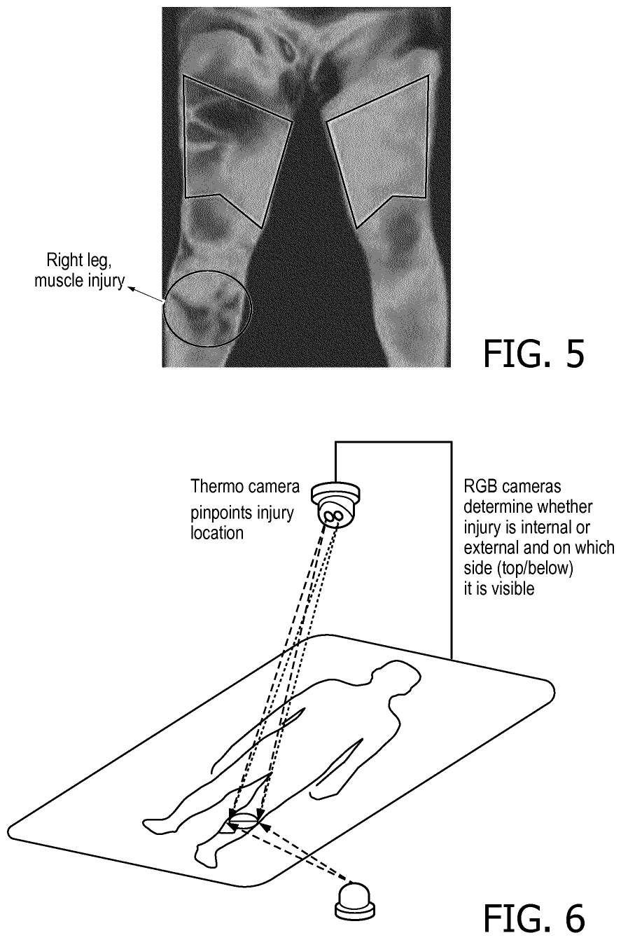

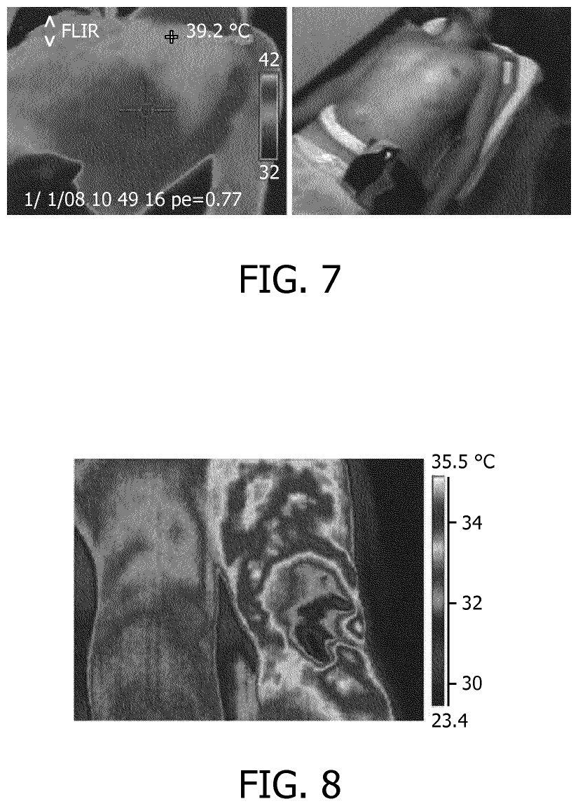

https://healthmanagement.org/c/imaging/news/infrared-thermography-offers-faster-diagnosis-of-orthopaedic-injuries;
https://researchportal.port.ac.uk/portal/files/5878124/thesis_for_Binding_final_copy_.3.pdf;
https://www.sciencedaily.com/releases/2015/11/151125083938.htm;
https://hackaday.com/2017/07/01/using-a-thermal-camera-to-spot-a-broken-wrist/
http://www.thermografie-centrum.nl/forms/m_infrared_imaging_for_emergency_medical_services.pdf; and
https://www.ncbi.nlm.nih.gov/pmc/articles/PMC4361423/. In some embodiments, the location of injury exhibits higher tissue temperature as illustrated in FIG. 5.

In some embodiments, system 100 is configured to distinguish internal injuries from external injuries. In some embodiments, system 100 is configured to perform a second scan using two normal RGB cameras 206, 212 at the site of the injury (i.e., determined using thermography techniques) to determine if the injury is external. In some embodiments, it is determined that injury is external by detecting wounds (i.e., blood) in the video images captured (see FIG. 6). In some embodiments, using two RGB cameras 206, 212 positioned to view the patient from top and below, makes it possible for system 100 to determine whether a potential open wound is located above or below on the body (see FIG. 6).

In some embodiments, injury scan and detection subsystem 124 is configured to determine and provide the following to the medical personnel 1) positon of the detected injury; 2) size of the detected injury; and 3) injury risk parameter. In some embodiments, the injury risk parameter is higher when the number of injuries are higher. In some embodiments, the injury risk parameter is higher when the size of the injuries are larger. In some embodiments, the injury risk parameter is higher when the number of injuries are higher and the size of the injuries are larger.

In some embodiments, emergency risk assessment system 120 is configured to assess the emergency risk of a patient based on input provided by 1) body contour detection subsystem 122; and 2) injury scan and detection subsystem 124 and to assist in the patient triage at the emergency department. In some embodiments, emergency risk assessment system 120 is configured to 1) assess the overall patient condition severity and emergency risk; 2) calculate projections regarding the patient expected deterioration rate in case no intervention is provided based on similar cases and patients. In some embodiments, emergency risk assessment system 120 is configured to compare and project the following emergency department triage patient data 1) injury position; 2) injury risk score; 3) injury severity; 4) thermographic scans; 5) size of injury; and 6) tissue temperature at location of injury. In some embodiments, emergency risk assessment system 120 is configured to advise optimal intervention timeframe and medical specialists needed for the case by using data including, but not limited to, 1) emergency department triage patient data (of similar patients cases); 2) previous intervention timeframe in similar cases; and 3) previous patient recovery data associated with the data collected at the emergency department triage. In some embodiments, emergency risk assessment system 120 is configured to update the triage list and inform medical staff of 1) patient current status and deterioration projection; 2) optimal intervention time; 3) update to triage list.

In some embodiments, healing progress monitoring subsystem 129 is configured to assess the healing progress of an injury detected and treated earlier. In some embodiments, healing progress monitoring subsystem 129 is configured to track over time the temperature trends at the injury location detected earlier. In some embodiments, for each scan, healing progress monitoring subsystem 129 is configured to determine the following: 1) the injury area, for example, based on the thermographic and the RGB scans; 2) the average temperature across the injury area; 3) the maximum temperature at the injury location (i.e., within the injury area); and 4) the spatial distribution of elevated temperatures within the injury area—quantified as the number of pixels corresponding to the elevated temperatures within the injury area. In some embodiments, healing progress monitoring subsystem 129 is configured to provide a healing progress indicator. In some embodiments, the healing progress indicator includes four components above and a healing score/parameter calculated as follows as a combination of the four components. In some embodiments, the healing score/parameter is calculated using the following equation (A) below.

HealingScore=InjuryArea⊗AvgTempInjuryArea⊗MaxTemp⊗DistributionTemp Equation (A)

In some embodiments, the healing score/parameter is calculated as below:

HealingScore=1/(w₁*InjuryArea+w₂*AvgTempInjuryArea+w₃*MaxTemp+w₄*DistributionTemp) Equation (B)

In some embodiments, w₁-w₄ in equation (B) are weights values that learned from patient population with a similar clinical profile and case as the patient at hand and are dependent on: 1) injury area; 2) patient clinical data (e.g., age, diabetes—coagulation, other conditions that might affect healing); and 3) injury external, internal or both.

In some embodiments, fracture scan and detection subsystem 118 is configured to automatically detect fractures. In some embodiments, system 100 is configured to 1) determine body contour of the patient using body contour detection subsystem 122; and 2) identify injuries; and 3) identify fractures In some embodiments, fracture scan and detection subsystem 118 uses IR camera 210 to compare left limb with right limb to detect anomalies in temperature. In some embodiments, if significant localized differences are detected in a limb compared to another, the presence of an injury is determined. In some embodiments, severity of injury is established based on area of elevated temperature and vital signs (e.g., temperature, heart rate).

In some embodiments, emergency risk assessment system 120 is configured to assess the emergency risk of a patient based on input provided by 1) body contour detection subsystem 122; 2) injury scan and detection subsystem 124; and 3) fracture scan and detection subsystem 118 and to assist in the patient triage at the emergency department.

In some embodiments, the probability/likelihood for limb fractures are assessed using the following procedures. In some embodiments, the patient's limbs are detected based on the data regarding the patient head position detected by body contour detection subsystem 122. In some embodiments, the injury presence positon is determined using the data put out by injury scan and detection subsystem 124. In some embodiments, the fracture presence is determined by fracture scan and detection subsystem 118. In some embodiments, for each injury position, soundwaves (as described in

http://www.techradar.com/news/world-of-tech/sound-waves-are-almost-as-good-as-x-rays-for-detecting-broken-bones-1293939) are used to determine if a fracture is potentially present at that location. In some embodiments, fracture scan and detection subsystem 118 is configured to use microwaves (see
https://www.ncbi.nlm.nih.gov/pmc/articles/PMC5047277/) to determine if a fracture is potentially present at that location.

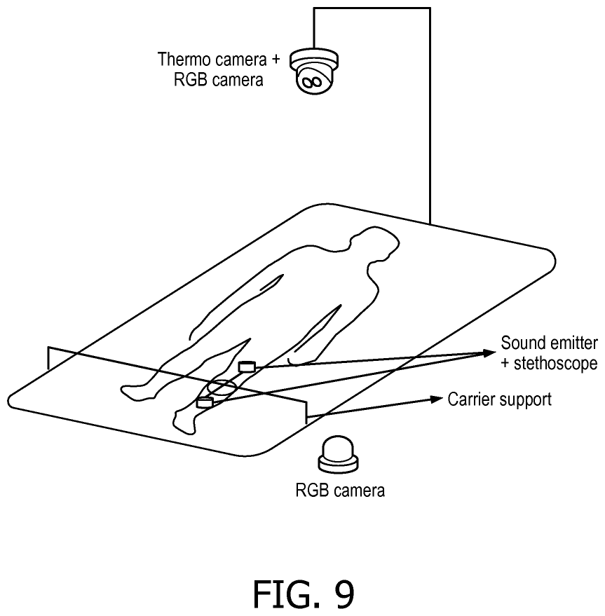

In some embodiments, system 100 is configured to execute the following procedures: 1) move support carrying the soundwave emitter to location of injury; 2) position sound emitter and audio sensor (e.g., stethoscope) at vertical extremities of the injury (see FIG. 9); 3) scan location of injury using sound waves; 4) move soundwave emitter horizontally to the other limb (same position) that is not injured; 5) scan location of without injury using sound waves; 6) compare the scan on top of position with no injury detected with the scan on top of position with injury detected

(https://www.obimed.com/wp-content/uploads/2016/05/A-Novel-Method-of-Detecting-Fractures-Via-Smartphone-final.pdf); and 7) determine the similarity level between the two scans.

In some embodiments, applying the two components (i.e., sound emitter and audio sensor (e.g., stethoscope)) can be done mechanically (as illustrated in the FIG. 9) or as an option this action can be performed by a nurse. In some embodiments, the support carrying the soundwave emitter is configured to be moveable on the whole length of the body, and/or placed above screen 202.

In some embodiments, in case an injury detected at the same position on the other limb, move sound emitter on the same limb higher up or lower down on a position where no injury is detected. In some embodiments, system 100 is configured to use a reference database of normal bone signals for comparison to injured limbs. In some embodiments, the limb selected for comparison belongs to individuals of similar physical characteristics with the patient (e.g., gender, age, height).

In some embodiments, the similarity level between the two scans (i.e., signal 1 and signal 2) is determined by fracture scan and detection subsystem 118 using equation (C).

ScansSimilarity=absolute(signal 1−signal 2) Equation (C)

If one of the captures (i.e., signal 1 or signal 2) is below the selected 3.5 dB/Hz threshold of vibrational energy (dB/Hz) compared to the other capture (i.e., signal 1 or signal 2), system 100 diagnoses the lower value capture (i.e., signal 1 or signal 2) as a fracture.

In some embodiments, fracture scan and detection subsystem 118 is configured to determine the probability for fracture based on equation (D).

FractureRisk=1/ScansSimilarity Equation (D)

In some embodiments, fracture scan and detection subsystem 118 is configured to provide 1) fracture position—indicated on a thermographic picture/image captured; and 2) fracture risk score (i.e., probability for fracture).

In some embodiments, fracture scan and detection subsystem 118 is configured to determine and provide the following to the medical personnel 1) positon of the detected fracture; 2) size of the detected fracture; and 3) fracture risk parameter. In some embodiments, fracture risk parameter is higher when the localized temperature (i.e., at the injury site) is higher. In some embodiments, fracture risk parameter is higher when the fracture size is larger. In some embodiments, fracture risk parameter is higher when the fracture size is larger and when the localized temperature (i.e., at the injury/fracture site) is higher. In some embodiments, fracture severity is determined by the size of elevated temperature in the thermal scan. In some embodiments, larger the size and the higher the temperature at the injury/fracture site could indicate sepsis and profuse haemorrhage associated with significant health risks.

In some embodiments, emergency risk assessment system 120 is configured to compare and project the following emergency department triage patient data 1) fracture position; 2) fracture risk score; 3) fracture severity; 4) thermographic scans; 5) size of injury/fracture; and 6) tissue temperature at location of injury/fracture. In some embodiments, emergency risk assessment system 120 is configured to advise optimal intervention timeframe and medical specialists needed for the case by using data including, but not limited to, 1) emergency department triage patient data (of similar patients cases); 2) previous intervention timeframe in similar cases; and 3) previous patient recovery data associated with the data collected at the emergency department triage. In some embodiments, emergency risk assessment system 120 is configured to update the triage list and inform medical staff of 1) patient current status and deterioration projection; 2) optimal intervention time; 3) update to triage list.

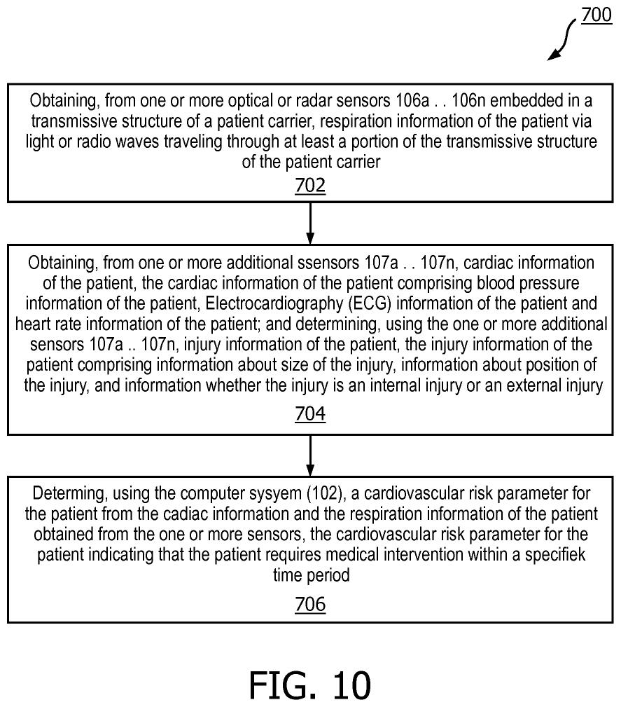

Referring to FIG. 10, a method 700 for assessing emergency risk of patients in an emergency department is provided. Method 700 is implemented by computer system 102 that comprises one or more physical processors executing computer program instructions that, when executed, perform method 700. In some embodiments, method 700 comprises: obtaining, from one or more optical or radar sensors 106a . . . 106n embedded in a transmissive structure of a patient carrier, respiration information of the patient via light or radio waves traveling through at least a portion of the transmissive structure of the patient carrier, the patient carrier being configured to support a patient to lay over the transmissive structure of the patient carrier, at procedure 702; obtaining, from one or more additional sensors 107a . . . 107n, cardiac information of the patient, the cardiac information of the patient including blood pressure information of the patient, Electrocardiography (ECG) information of the patient and/or heart rate information of the patient at procedure 704; determining, using the one or more additional sensors 107a . . . 107n, injury information of the patient, the injury information of the patient comprising information about size of the injury, information about position of the injury, and/or information about whether the injury is an internal injury or an external injury; and determining, using computer system 102, an emergency risk parameter for the patient based on the cardiac information, the injury information and the respiration information of the patient obtained from one or more sensors 106a . . . 106n; 107a . . . 107n, the emergency risk parameter for the patient indicating that the patient requires medical intervention within a specified time period at procedure 706.

In some embodiments, computer system 102 is further configured to determine projected deterioration rate information for the patient in case no medical intervention is provided within the specified time period using previously determined emergency risk parameters of similar patients. In some embodiments, computer system 102 is further configured to calculate projections regarding the patient expected deterioration rate in case no medical intervention is provided based on similar cases and patients.

In some embodiments, computer system 102 is configured to notify a clinician of the determined emergency risk parameter for the patient, the specified time period for the medical intervention, and the determined projected deterioration rate information for the patient. In some embodiments, when the determined emergency risk parameter indicates a patient needs medical attention within the specified time period, computer system 102 is configured to generate audio and/or visuals alerts and/or messages notifying clinicians thereof. It is contemplated that such a message can be provided to the clinicians via the communication network 150. In some embodiments, computer system 102 is also configured to notify only (and all) medical specialists needed for the case. In some embodiments, computer system 102 is configured to notify a clinician of 1) optimal intervention timeframe, and 2) the overall patient condition severity and emergency risk.

In some embodiments, computer system 102 is configured to update the triage list in real time. In some embodiments, computer system 102 is configured to dynamically update the triage list. In some embodiments, computer system 102 is configured to update the triage list to include 1) optimal intervention timeframe, and 2) the overall patient condition severity and emergency risk. In some embodiments, computer system 102 is configured to enable the medical personnel or clinician to prioritize patients that are most critical.

In some embodiments, the various computers and subsystems illustrated in FIG. 1 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information or data. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the system 100. As such, the processors may include one or more of a digital processor, an analog processor, or a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-124 and 129 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the subsystems 112-124 and 129 described herein is for illustrative purposes, and is not intended to be limiting, as subsystems 112-124 and 129 may provide more or less functionality than is described. As another example, additional subsystems may be programmed to perform some or all of the functionality attributed herein to subsystems 112-124 and 129.

In some embodiments, system 100 may also include a communication interface that is configured to send the determined emergency risk assessment through an appropriate wireless communication method (e.g., Wi-Fi, Bluetooth, internet, etc.) to necessary medical or clinical personnel or systems for further processing. In some embodiments, system 100 may include a recursive tuning subsystem that is configured to recursively tune its intelligent decision making subsystem using available data or information to provide better overall cardiovascular risk assessment. In some embodiments, intelligent decision making subsystem, communication interface and recursive tuning subsystem may be part of computer system (comprising server 102).

In some embodiments, a subsystem of system 100 is configured to continuously obtain subsequent patient cardiac information, respiratory information, body contour information, injury risk information, fracture information, stroke risk parameter, respiratory risk parameter, cardiovascular risk parameter and/or emergency risk parameter. That is, the subsystem may continuously obtain subsequent patient cardiac information, respiratory information, body contour information, injury risk information, fracture information, stroke risk parameter, respiratory risk parameter, cardiovascular risk parameter and/or emergency risk parameter. As an example, the subsequent information may comprise additional information corresponding to a subsequent time (after a time corresponding to information that was used to determine the emergency risk parameter). The subsequent information may be utilized to further update or modify the emergency risk parameter (e.g., new information may be used to dynamically update or modify the emergency risk parameter), etc. For example, the subsequent information may also be configured to provide further input to determine the emergency risk parameter. In some embodiments, a subsystem of system 100 may be configured to determine the emergency risk parameter in accordance with a recursively refined profile (e.g., refined through recursive application of profile refinement algorithms) based on previously collected or subsequent patient health information.

In some embodiments, intelligent decision making subsystem may be a machine learning algorithm or method that is used for combining different data sources and intelligent decision making. In some embodiments, the machine learning algorithm of intelligent decision making subsystem may include time-varying algorithm that may model the changes of parameters and their relationship over time. For example, in some embodiments, the machine learning algorithm of intelligent decision making subsystem may include Hidden Markov models or Dynamic Bayesian networks. In some embodiments, the machine learning algorithm of intelligent decision making subsystem may include non-time varying models. For example, the machine learning algorithm of intelligent decision making subsystem may include a classifier such as support vector machines or Naïve Bayes. In some embodiments, the machine learning algorithm of intelligent decision making subsystem may include modelling previous operations and learning from data or information. There are several sources of information that are used as inputs to intelligent decision making subsystem so as to get more certainty in the emergency risk assessment score. Intelligent decision making subsystem combines inputs from these several sources.

The present patent application is used in the healthcare domain.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the present patent application has been described in detail for the purpose of illustration based on what is currently considered to be the most practical and preferred embodiments, it is to be understood that such detail is solely for that purpose and that the present patent application is not limited to the disclosed embodiments, but, on the contrary, is intended to cover modifications and equivalent arrangements that are within the spirit and scope of the appended claims. For example, it is to be understood that the present patent application contemplates that, to the extent possible, one or more features of any embodiment can be combined with one or more features of any other embodiment.

1. A system for assessing emergency risk of patients in an emergency department, the system comprising:
a patient carrier comprising a transmissive structure, the patient carrier being configured to support a patient to lay over the transmissive structure of the patient carrier;
one or more optical or radar sensors being configured to measure respiration information of the patient via light or radio waves traveling through at least a portion of the transmissive structure of the patient carrier;
one or more additional sensors configured to
1. measure cardiac information of the patient, the cardiac information of the patient comprising blood pressure information of the patient, Electrocardiography (ECG) information of the patient, and/or heart rate information of the patient, and
2. obtain data relating to the patient, from which injury information of the patient can be determined, the injury information of the patient comprising information about size of the injury, information about position of the injury, and/or information about whether the injury is an internal injury or an external injury; and
a computer system comprising one or more physical processors programmed with computer program instructions that, when executed, cause the computer system to:
determine an emergency risk parameter for the patient based upon the cardiac information, the respiration information and the injury information of the patient obtained from the one or more sensors, the emergency risk parameter for the patient indicating that the patient requires medical intervention within a specified time period.

2. The system of claim 1, wherein the one or more optical or radar sensors comprises one or more radar sensors (i) embedded in the transmissive portion of the patient carrier and (ii) configured to measure the respiration information of the patient via radio waves traveling through at least a portion of the transmissive structure of the patient carrier.

3. The system of claim 1, wherein the one or more optical or radar sensors comprises one or more optical sensors (i) embedded in the transmissive portion of the patient carrier and (ii) configured to measure the respiration information of the patient via light traveling through at least a portion of the transmissive structure of the patient carrier.

4. The system of claim 1, wherein the computer system is further configured to:
determine a cardiovascular risk parameter for the patient using the blood pressure information of the patient obtained from a blood pressure sensor, the Electrocardiography (ECG) information of the patient obtained from an Electrocardiography (ECG) sensor, the respiration information of the patient obtained from a respiration sensor and the heart rate information of the patient obtained from a heart rate sensor;
determine an injury risk parameter for the patient using body contour information obtained from a thermal camera, the injury information of the patient obtained from the thermal camera, an image sensor and an audio sensor;
determine a respiratory risk parameter for the patient using the one or more optical or radar sensors, the thermal camera, and the image sensor;
wherein the one or more optical or radar sensors comprises the respiration sensor, and the one or more additional sensors comprises the blood pressure sensor, the Electrocardiography (ECG) sensor, the heart rate sensor, the thermal camera, the image sensor, and the audio sensor; and
determine the emergency risk parameter for the patient using the determined cardiovascular risk parameter, the determined injury risk parameter, and the determined respiratory risk parameter for the patient.

5. The system of claim 1, wherein the computer system is further configured to determine projected deterioration rate information for the patient in case no medical intervention is provided within the specified time period using previously determined emergency risk parameters of similar patients.

6. The system of claim 5, wherein the computer system is further configured to notify a clinician of the determined emergency risk parameter for the patient, the specified time period for the medical intervention, and the determined projected deterioration rate information for the patient.

7. A method for assessing emergency risk of patients in an emergency department, the method being implemented by a computer system that comprises one or more physical processors executing computer program instructions that, when executed, perform the method, the method comprising:
obtaining, from one or more optical or radar sensors disposed in a transmissive structure of a patient carrier, respiration information of the patient via light or radio waves traveling through at least a portion of the transmissive structure of the patient carrier, the patient carrier being configured to support a patient to lay over the transmissive structure of the patient carrier;
obtaining, from one or more additional sensors, cardiac information of the patient, the cardiac information of the patient comprising blood pressure information of the patient, Electrocardiography (ECG) information of the patient and/or heart rate information of the patient;
obtaining, using the one or more additional sensors, data relating to the patient, from which injury information of the patient can be determined, the injury information of the patient comprising information about size of the injury, information about position of the injury, and/or information about whether the injury is an internal injury or an external injury; and
determining, using the computer system, an emergency risk parameter for the patient based on the cardiac information, the respiration information and the injury information of the patient obtained from the one or more sensors, the emergency risk parameter for the patient indicating that the patient requires medical intervention within a specified time period.

8. The method of claim 7, wherein the optical or radar sensors comprises one or more radar sensors (i) embedded in the transmissive portion of the patient carrier and (ii) configured to measure the respiration information of the patient via radio waves traveling through at least a portion of the transmissive structure of the patient carrier.

9. The method of claim 7, wherein the one or more optical or radar sensors comprises one or more optical sensors (i) embedded in the transmissive portion of the patient carrier and (ii) configured to measure the respiration information of the patient via light traveling through at least a portion of the transmissive structure of the patient carrier.

10. The method of claim 7, further comprising:
determining, using the computer system, a cardiovascular risk parameter for the patient using the blood pressure information of the patient obtained from a blood pressure sensor, the Electrocardiography (ECG) information of the patient obtained from an Electrocardiography (ECG) sensor, the respiration information of the patient obtained from a respiration sensor and the heart rate information of the patient obtained from a heart rate sensor;
determining, using the computer system, an injury risk parameter for the patient using body contour information obtained from a thermal camera, the injury information of the patient obtained from the thermal camera, an image sensor and an audio sensor;
determining, using the computer system, a respiratory risk parameter for the patient using the one or more optical or radar sensors, the thermal camera, and the image sensor;
wherein the one or more optical or radar sensors comprises the respiration sensor, and the one or more additional sensors comprises the blood pressure sensor, the Electrocardiography (ECG) sensor, the heart rate sensor, the thermal camera, the image sensor, and the audio sensor; and
determining, using the computer system, the emergency risk parameter for the patient using the determined cardiovascular risk parameter, the determined injury risk parameter, and the determined respiratory risk parameter for the patient.

11. he method of claim 7, further comprising determining, using the computer system, projected deterioration rate information for the patient in case no medical intervention is provided within the specified time period using previously determined emergency risk parameters of similar patients.

12. The method of claim 11, further comprising notifying, using the computer system, a clinician of the determined emergency risk parameter for the patient, the specified time period for the medical intervention, and the determined projected deterioration rate information for the patient.

13. A system for assessing emergency risk of patients in an emergency department, the system comprising:
a means for executing machine-readable instructions with at least one processor, wherein the machine-readable instructions comprising:
obtaining, from one or more optical or radar sensors disposed in a transmissive structure of a patient carrier, respiration information of the patient via light or radio waves traveling through at least a portion of the transmissive structure of the patient carrier, the patient carrier being configured to support a patient to lay over the transmissive structure of the patient carrier;
obtaining, from one or more additional sensors, cardiac information of the patient, the cardiac information of the patient comprising blood pressure information of the patient, Electrocardiography (ECG) information of the patient and/or heart rate information of the patient;
obtaining, using the one or more additional sensors, injury information of the patient, the injury information of the patient comprising information about size of the injury, information about position of the injury, and/or information about whether the injury is an internal injury or an external injury; and
determining, using the computer system, an emergency risk parameter for the patient based on the cardiac information, the respiration information and the injury information of the patient obtained from the one or more sensors, the emergency risk parameter for the patient indicating that the patient requires medical intervention within a specified time period.

14. The system of claim 13, wherein the one or more optical or radar sensors comprises one or more radar sensors (i) embedded in the transmissive portion of the patient carrier and (ii) configured to measure the respiration information of the patient via radio waves traveling through at least a portion of the transmissive structure of the patient carrier.

15. The system of claim 13, wherein the one or more optical or radar sensors comprises one or more optical sensors (i) embedded in the transmissive portion of the patient carrier and (ii) configured to measure the respiration information of the patient via light traveling through at least a portion of the transmissive structure of the patient carrier.

16. The system of claim 13, wherein the machine-readable instructions further comprising:
determining, using the computer system, a cardiovascular risk parameter for the patient using the blood pressure information of the patient obtained from a blood pressure sensor, the Electrocardiography (ECG) information of the patient obtained from an Electrocardiography (ECG) sensor, the respiration information of the patient obtained from a respiration sensor and the heart rate information of the patient obtained from a heart rate sensor;
determining, using the computer system, an injury risk parameter for the patient using body contour information obtained from a thermal camera, the injury information of the patient obtained from the thermal camera, an image sensor and an audio sensor;
determining, using the computer system, a respiratory risk parameter for the patient using the one or more optical or radar sensors, the thermal camera, and the image sensor;
wherein the one or more optical or radar sensors comprises the respiration sensor, and the one or more additional sensors comprises the blood pressure sensor, the Electrocardiography (ECG) sensor, the heart rate sensor, the thermal camera, the image sensor, and the audio sensor; and
determining, using the computer system, the emergency risk parameter for the patient using the determined cardiovascular risk parameter, the determined injury risk parameter, and the determined respiratory risk parameter for the patient.

17. The system of claim 13, wherein the machine-readable instructions further comprising: determining projected deterioration rate information for the patient in case no medical intervention is provided within the specified time period using previously determined emergency risk parameters of similar patients.

18. The system of claim 17, wherein the machine-readable instructions further comprising: notifying a clinician of the determined emergency risk parameter for the patient, the specified time period for the medical intervention, and the determined projected deterioration rate information for the patient.
